SEQUENCE LISTING (38 sequences)

SEQ ID NO: 1 - DNA, 321 bp, Artificial Sequence
  M-spacer
ggccagcctagagaaccccaggtgtacaccctgcctcccagccaggaagagatgaccaagaaccaggtgtccctgacctgcctggtcaaaggcttctaccccagcgatatcgccgtggaatgggagagcaacggccagcccgagaacaactacaagaccaccccccctgtgctggacagcgacggcagcttcttcctgtactcccggctgaccgtggacaagagccggtggcaggaaggcaacgtcttcagctgcagcgtgatgcacgaggccctgcacaaccactacacccagaagtccctgagcctgagcctgggcaag

SEQ ID NO: 2 - DNA, 651 bp, Artificial Sequence
  L-spacer
gcccccgagttcgacggcggacccagcgtgttcctgttcccccccaagcccaaggacaccctgatgatcagccggacccccgaggtgacctgcgtggtggtggacgtgagccaggaagatcccgaggtccagttcaattggtacgtggacggcgtggaagtgcacaacgccaagaccaagcccagagaggaacagttccagagcacctaccgggtggtgtctgtgctgaccgtgctgcaccaggactggctgaacggcaaagaatacaagtgcaaggtgtccaacaagggcctgcccagcagcatcgaaaagaccatcagcaaggccaagggccagcctcgcgagccccaggtgtacaccctgcctccctcccaggaagagatgaccaagaaccaggtgtccctgacctgcctggtgaagggcttctaccccagcgacatcgccgtggagtgggagagcaacggccagcctgagaacaactacaagaccacccctcccgtgctggacagcgacggcagcttcttcctgtacagccggctgaccgtggacaagagccggtggcaggaaggcaacgtctttagctgcagcgtgatgcacgaggccctgcacaaccactacacccagaagagcctgagcctgtccctgggcaag

SEQ ID NO: 3 - protein, 90 aa, Artificial Sequence [sequence omitted]

SEQ ID NO: 4 - protein, 196 aa, Artificial Sequence
  L-spacer
MISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 5 - protein, 117 aa, Artificial Sequence
  Anti-CD33 VH domain
QVQLVQSGAEVKKPGASVKVSCKASGYTFTNYDINWVRQAPGQGLEWIGWIYPGDGSTKYNEKFKAKATLTADTSTSTAYMELRSLRSDDTAVYYCASGYEDAMDYWGQGTTVTVSS

SEQ ID NO: 6 - protein, 5 aa, Artificial Sequence
  VH-CDR1
NYDIN

SEQ ID NO: 7 - protein, 17 aa, Artificial Sequence
  VH-CDR2
WIYPGDGSTKYNEKFKA

SEQ ID NO: 8 - protein, 8 aa, Artificial Sequence
  VH-CDR3
GYEDAMDY

SEQ ID NO: 9 - protein, 107 aa, Artificial Sequence
  Anti-CD33 VL domain
DIQMTQSPSSLSASVGDRVTINCKASQDINSYLSWFQQKPGKAPKTLIYRANRLVDGVPSRFSGSGSGQDYTLTISSLQPEDFATYYCLQYDEFPLTFGGGTKVEIK

SEQ ID NO: 10 - protein, 11 aa, Artificial Sequence
  VL-CDR1
KASQDINSYLS

SEQ ID NO: 11 - protein, 8 aa, Artificial Sequence
  VL-CDR2
RANNRLVD

SEQ ID NO: 12 - protein, 9 aa, Artificial Sequence
  VL-CDR3
LQYDEFPLT

SEQ ID NO: 13 - protein, 15 aa, Artificial Sequence
  scFv linker
GGGGSGGGGSGGGGS

SEQ ID NO: 14 - protein, 12 aa, Artificial Sequence
  S spacer
ESKYGPPCPPCP

SEQ ID NO: 15 - DNA, 36 bp, Artificial Sequence
  S spacer
gaatctaagtacggaccgccctgccccccttgccct

SEQ ID NO: 16 - protein, 119 aa, Artificial Sequence
  M spacer
ESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 17 - DNA, 357 bp, Artificial Sequence
  M spacer
gaatctaagtacggaccgccctgccccccttgccctggccagcctagagaaccccaggtgtacaccctgcctcccagccaggaagagatgaccaagaaccaggtgtccctgacctgcctggtcaaaggcttctaccccagcgatatcgccgtggaatgggagagcaacggccagcccgagaacaactacaagaccaccccccctgtgctggacagcgacggcagcttcttcctgtactcccggctgaccgtggacaagagccggtggcaggaaggcaacgtcttcagctgcagcgtgatgcacgaggccctgcacaaccactacacccagaagtccctgagcctgagcctgggcaag

SEQ ID NO: 18 - protein, 229 aa, Artificial Sequence
  L spacer
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 19 - DNA, 685 bp, Artificial Sequence
  L spacer
atctaagtacggaccgccctgccccccttgccctgcccccgagttcgacggcggacccagcgtgttcctgttcccccccaagcccaaggacaccctgatgatcagccggacccccgaggtgacctgcgtggtggtggacgtgagccaggaagatcccgaggtccagttcaattggtacgtggacggcgtggaagtgcacaacgccaagaccaagcccagagaggaacagttccagagcacctaccgggtggtgtctgtgctgaccgtgctgcaccaggactggctgaacggcaaagaatacaagtgcaaggtgtccaacaagggcctgcccagcagcatcgaaaagaccatcagcaaggccaagggccagcctcgcgagccccaggtgtacaccctgcctccctcccaggaagagatgaccaagaaccaggtgtccctgacctgcctggtgaagggcttctaccccagcgacatcgccgtggagtgggagagcaacggccagcctgagaacaactacaagaccacccctcccgtgctggacagcgacggcagcttcttcctgtacagccggctgaccgtggacaagagccggtggcaggaaggcaacgtctttagctgcagcgtgatgcacgaggccctgcacaaccactacacccagaagagcctgagcctgtccctgggcaag

SEQ ID NO: 20 - protein, 28 aa, Artificial Sequence
  CD28tm
MFWVLVVVGGVLACYSLLVTVAFIIFWV

SEQ ID NO: 21 - DNA, 84 bp, Artificial Sequence
  CD28tm
atgttctgggtgctggtggtggtcggaggcgtgctggcctgctacagcctgctggtcaccgtggccttcatcatcttttgggtg

SEQ ID NO: 22 - DNA, 126 bp, Artificial Sequence
  41-BB
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 23 - DNA, 336 bp, Artificial Sequence
  CD3zeta
cgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaagg

SEQ ID NO: 24 - DNA, 72 bp, Artificial Sequence
  T2A
ctcgagggcggcggagagggcagaggaagtcttctaacatgcggtgacgtggaggagaatcccggccctagg

SEQ ID NO: 25 - DNA, 1005 bp, Artificial Sequence
  EGFRt
cgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatg

SEQ ID NO: 26 - protein, 187 aa, Artificial Sequence
  DHFRdm
MVGSLNCIVAVSQNMGIGKNGDFPWPPLRNESRYFQRMTTTSSVEGKQNLVIMGKKTWFSIPEKNRPLKGRINLVLSRELKEPPQGAHFLSRSLDDALKLTEQPELANKVDMVWIVGGSSVYKEAMNHPGHLKLFVTRIMQDFESDTFFPEIDLEKYKLLPEYPGVLSDVQEEKGIKYKFEVYEKND

SEQ ID NO: 27 - DNA, 564 bp, Artificial Sequence
  DHFRdm
atggttggttcgctaaactgcatcgtcgctgtgtcccagaacatgggcatcggcaagaacggggacttcccctggccaccgctcaggaatgaatccagatatttccagagaatgaccacaacctcttcagtagaaggtaaacagaatctggtgattatgggtaagaagacctggttctccattcctgagaagaatcgacctttaaagggtagaattaatttagttctcagcagagaactcaaggaacctccacaaggagctcattttctttccagaagtctagatgatgccttaaaacttactgaacaaccagaattagcaaataaagtagacatggtctggatagttggtggcagttctgtttataaggaagccatgaatcacccaggccatcttaaactatttgtgacaaggatcatgcaagactttgaaagtgacacgttttttccagaaattgatttggagaaatataaacttctgccagaatacccaggtgttctctctgatgtccaggaggagaaaggcattaagtacaaatttgaagtatatgagaagaatgattaa

SEQ ID NO: 28 - protein, 15 aa, Artificial Sequence
  CD19 signal sequence
MPPPRLLFFLLFLTP

SEQ ID NO: 29 - protein, 22 aa, Artificial Sequence
  GMCSFR signal sequence
MLLLVTSLLLCELPHPAFLLIP

SEQ ID NO: 30 - DNA, 972 bp, Artificial Sequence
  CD19t
atgccacctcctcgcctcctcttcttcctcctcttcctcacccccatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggccatctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtgtcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttcaaagagccctggtcctgaggaggaaaagataa

SEQ ID NO: 31 - protein, 323 aa, Artificial Sequence
  CD19t
MPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCVPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKR

SEQ ID NO: 32 - DNA, 66 bp, Artificial Sequence
  GM-CSF receptor-chain signal sequence
atgcttctcctggtgacaagccttctgctctgtgagttaccacacccagcattcctcctgatccca

SEQ ID NO: 33 - protein, 22 aa, Artificial Sequence
  GM-CSF receptor-chain signal sequence
MLLLVTSLLLCELPHPAFLLIP

SEQ ID NO: 34 - DNA, 2175 bp, Artificial Sequence
  pJ03975_CD33VH-VL-IgG4Hinge-CD28tm-4-1BB-T2A- EGFRt_epHIV72
atgcttctcctggtgacaagccttctgctctgtgagttaccacacccagcattcctcctgatcccacaggttcagctggtgcagtctggagctgaggtgaagaagcctggggcctcagtgaaggtctcctgcaaggcttctggttacacctttaccaattatgatataaattgggtgagacaggcccctggacaagggcttgagtggattggatggatttatcctggagatggtagtaccaaatataatgagaaattcaaggccaaggctaccctgacagctgacacatccaccagcacagcctacatggagctgaggagcctgagatctgatgacacagctgtgtattactgtgcttctggatatgaagatgctatggactactgggggcaagggaccacagtcacagtctcctcaggtggcggtggcagcggcggtggtggttccggaggcggcggttctgacatccagatgacccagtctccatcctcactgtctgcatctgtaggagacagagtcaccatcaattgtaaggctagtcaggacattaatagctatttgagctggtttcagcagaaaccagggaaagcccctaagaccctgatctatagagcaaatagattggtagatggggtcccatcaaggttctctggcagtggatctgggcaagattatactctcaccatcagcagcctgcagcctgaagattttgcaacttattactgcttgcagtatgatgagtttcctctcacatttggaggagggaccaaggtggagatcaaagaatctaagtacggaccgccctgccccccttgccctatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgctcgagggcggcggagagggcagaggaagtcttctaacatgcggtgacgtggaggagaatcccggccctaggatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtga

SEQ ID NO: 35 - protein, 724 aa, Artificial Sequence
  pJ03975_CD33VH-VL-IgG4Hinge-CD28tm-4-1BB-T2A- EGFRt_epHIV72
MLLLVTSLLLCELPHPAFLLIPQVQLVQSGAEVKKPGASVKVSCKASGYTFTNYDINWVRQAPGQGLEWIGWIYPGDGSTKYNEKFKAKATLTADTSTSTAYMELRSLRSDDTAVYYCASGYEDAMDYWGQGTTVTVSSGGGGSGGGGSGGGGSDIQMTQSPSSLSASVGDRVTINCKASQDINSYLSWFQQKPGKAPKTLIYRANRLVDGVPSRFSGSGSGQDYTLTISSLQPEDFATYYCLQYDEFPLTFGGGTKVEIKESKYGPPCPPCPMFWVLVVVGGVLACYSLLVTVAFIIFWVKRGRKKLLYIFKQPFMRPVQTTQEEDGCSCRFPEEEEGGCELLEGGGEGRGSLLTCGDVEENPGPRMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 36 - DNA, 2616 bp, Artificial Sequence
  pJ04043-CD33scFv-VH-VL-CD8hinge-tm-41BB-zeta- T2A-EGFRt-epHIV72
atgcttctcctggtgacaagccttctgctctgtgagttaccacacccagcattcctcctgatcccacaggttcagctggtgcagtctggagctgaggtgaagaagcctggggcctcagtgaaggtctcctgcaaggcttctggttacacctttaccaattatgatataaattgggtgagacaggcccctggacaagggcttgagtggattggatggatttatcctggagatggtagtaccaaatataatgagaaattcaaggccaaggctaccctgacagctgacacatccaccagcacagcctacatggagctgaggagcctgagatctgatgacacagctgtgtattactgtgcttctggatatgaagatgctatggactactgggggcaagggaccacagtcacagtctcctcaggtggcggtggcagcggcggtggtggttccggaggcggcggttctgacatccagatgacccagtctccatcctcactgtctgcatctgtaggagacagagtcaccatcaattgtaaggctagtcaggacattaatagctatttgagctggtttcagcagaaaccagggaaagcccctaagaccctgatctatagagcaaatagattggtagatggggtcccatcaaggttctctggcagtggatctgggcaagattatactctcaccatcagcagcctgcagcctgaagattttgcaacttattactgcttgcagtatgatgagtttcctctcacatttggaggagggaccaaggtggagatcaaagaatctaagtacggaccggccaagcctaccaccacccctgcccctagacctccaacacccgccccaacaatcgccagccagcctctgtctctgaggcccgaggcttgtagaccagctgctggcggagccgtgcacaccagaggactggatttcgcctgcgacatctacatctgggcccctctggccggcacatgtggcgtgctgctgctgagcctcgtgatcaccaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggctcgagggcggcggagagggcagaggaagtcttctaacatgcggtgacgtggaggagaatcccggccctaggatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtga

SEQ ID NO: 37 - protein, 871 aa, Artificial Sequence
  pJ04043-CD33scFv-VH-VL-CD8hinge-tm-41BB-zeta- T2A-EGFRt-epHIV72
MLLLVTSLLLCELPHPAFLLIPQVQLVQSGAEVKKPGASVKVSCKASGYTFTNYDINWVRQAPGQGLEWIGWIYPGDGSTKYNEKFKAKATLTADTSTSTAYMELRSLRSDDTAVYYCASGYEDAMDYWGQGTTVTVSSGGGGSGGGGSGGGGSDIQMTQSPSSLSASVGDRVTINCKASQDINSYLSWFQQKPGKAPKTLIYRANRLVDGVPSRFSGSGSGQDYTLTISSLQPEDFATYYCLQYDEFPLTFGGGTKVEIKESKYGPAKPTTTPAPRPPTPAPTIASQPLSLRPEACRPAAGGAVHTRGLDFACDIYIWAPLAGTCGVLLLSLVITKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 38 - protein, 71 aa, Artificial Sequence
  CD8a hinge and TM
GPAKPTTTPAPRPPTPAPTIASQPLSLRPEACRPAAGGAVHTRGLDFACDIYIWAPLAGTCGVLLLSLVIT

(19) United States
(12) Patent Application Publication
(10) Pub. No.: US 2022/0195441 A1
(43) Pub. Date: Jun. 23, 2022

(54) COMBINATORIAL CAR T CELL AND HEMATOPOEITIC STEM CELL GENETIC ENGINEERING FOR SPECIFIC IMMUNOTHERAPY OF MYELOID LEUKEMIAS

(71) Applicants: Seattle Children's Hospital (dba Seattle Children's Research Institute), Seattle, WA (US); Fred Hutchinson Cancer Research Center, Seattle, WA (US)

(72) Inventors: Michael C. Jensen, Seattle, WA (US); Soheil Meshinchi, Seattle, WA (US)

(21) Appl. No.: 17/594,542

(22) PCT Filed: Apr. 21, 2020
(86) PCT No.: PCT/US2020/029083

§ 371 Date: Oct. 21, 2021

Related U.S. Application Data
(60) Provisional application No. 62/838,808, filed on Apr. 25, 2019.

(51) Int. Cl. C12N 15/62; C07K 16/28; C07K 14/705; C07K 14/725; C07K 14/71

(52) U.S. Cl. CPC C12N 15/625; C07K 16/2803; C07K 14/70578; C07K 14/7051; A61K 2039/505; C07K 14/70517; C07K 14/70521; C07K 2319/30; C07K 2319/33; C07K 14/71

(57) ABSTRACT

Some embodiments of the methods and compositions provided herein include chimeric antigen receptors (CAR)s which specifically bind to an epitope of CD33, such as an epitope encoded by exon 2 of CD33. Some embodiments include the use of such CARs for effective and safe therapies for myeloid leukemias, such as acute myeloid leukemia and chronic myeloid leukemia.

### CROSS-REFERENCE TO RELATED APPLICATIONS

This application claims priority to U.S. Prov. App. No. 62/838,808 filed Apr. 25, 2019 entitled “COMBINATORIAL CAR T CELL AND HEMATOPOEITIC STEM CELL GENETIC ENGINEERING FOR SPECIFIC IMMUNOTHERAPY OF MYELOID LEUKEMIAS” which is expressly incorporated herein by reference in its entirety.

### REFERENCE TO SEQUENCE LISTING

The present application is being filed along with a Sequence Listing in electronic format. The Sequence Listing is provided as a file entitled SCRI122WOSEQLIST, created Apr. 14, 2020, which is approximately 42 Kb in size. The information in the electronic format of the Sequence Listing is incorporated herein by reference in its entirety.

### FIELD OF THE INVENTION

Some aspects of the methods and compositions provided herein relate to chimeric antigen receptors (CAR)s, which specifically bind to an epitope of CD33, such as an epitope encoded by exon 2 of CD33. Some embodiments include the use of such CARs for effective and safe therapies for treating, inhibiting, or ameliorating myeloid leukemias, such as acute myeloid leukemia or chronic myeloid leukemia.

### BACKGROUND OF THE INVENTION

Immunotherapy employing adoptive transfer of genetically modified T cells, which express chimeric antigen receptors specific for CD19, have resulted in breakthrough anti-leukemia effects in clinical trials involving patients with highly advanced CD19 expressing leukemias and lymphomas in adults and children. CD19 is a unique antigen that is pervasively expressed by B cell lineage hematologic neoplasms but also expressed on non-malignant B cells. The therapeutic ablation of all B cells (malignant and non-malignant) is tolerated by patients because the induced B cell aplasia and potential immunodeficiency is ameliorated by the persistence of antibody producing CD19− negative plasma cells and the availability of intravenous immunoglobulin (IVIG) replacement therapy. To date, a CD19 equivalent has not been identified for CAR T cell therapy of acute myeloid leukemia (AML) and targets under investigation, such as CD123, carry the risk of causing pancytopenias by cross targeting of hematopoietic stem cells or their early progenitors.

CD33 is a myeloid cell surface antigen that is also expressed on a high percentage of myelogenous leukemias and has been the target for antibody-based therapeutics, as well as CAR T cell specificity. CD33 is expressed by non-malignant myeloid derived cells that have roles for host protection from infectious organisms. Unlike CD19-expressing B cells, the replacement of myeloid cell protective function cannot be replaced by alternate CD33-nonexpressing cell sources. In addition, CD33 is expressed in myeloid progenitors and targeting CD33 by CAR T cell therapy runs the risk of causing pancytopenias or marrow ablation. Accordingly, alternative approaches to overcome these obstacles and generate CD33-based therapies is needed.

### SUMMARY OF THE INVENTION

Some embodiments of the methods and compositions provided herein include a nucleic acid encoding a chimeric antigen receptor (CAR), wherein the CAR comprises: a ligand binding domain capable of or configured to specifically bind to a long isoform of a CD33 protein (CD33M); a spacer; a transmembrane domain; and an intracellular signaling domain.

In some embodiments, the ligand binding domain is capable of or configured to specifically bind to an IgV domain of a CD33 protein.

In some embodiments, the ligand binding domain is capable of or configured to specifically binding to an epitope encoded by exon 2 of a CD33 gene.

In some embodiments, the ligand binding domain comprises a complementarity-determining region (CDR) comprising the amino acid sequence having 0-4 conservative amino acid substitutions of any one of SEQ ID NOs:06-12.

In some embodiments, the ligand binding domain comprises a VH domain comprising an amino acid sequence having at least 95% identity to the amino acid sequence of SEQ ID NO:05.

In some embodiments, the ligand binding domain comprises a VL domain comprising an amino acid sequence having at least 95% identity to the amino acid sequence of SEQ ID NO:09.

In some embodiments, the ligand binding domain comprises a VH domain and a VL domain, wherein a polynucleotide encoding the VH domain is 5′ of a polynucleotide encoding the VH domain.

In some embodiments, the ligand binding domain comprises a VH domain and a VL domain, wherein a polynucleotide encoding the VH domain is 3′ of a polynucleotide encoding the VH domain.

In some embodiments, the spacer comprises an IgG hinge or a CD8a domain.

In some embodiments, the spacer comprises an amino acid sequence having at least 95% identity to the amino acid sequence of any one of SEQ ID NOs: 14, 16 or 18.

In some embodiments, the spacer comprises less than 230 consecutive amino acids but not zero.

In some embodiments, the spacer comprises less than 120 consecutive amino acids but not zero.

In some embodiments, the spacer comprises less than 15 consecutive amino acids but not zero.

In some embodiments, the spacer comprises a de-immunized extracellular spacer.

In some embodiments, the ligand binding domain comprises a VH domain and a VL domain, wherein a polynucleotide encoding the VH domain is 5′ of a polynucleotide encoding the VH domain; the spacer comprises an amino acid sequence having at least 95% identity to the amino acid sequence of any one of SEQ ID NO:14; and the spacer comprises less than 15 consecutive amino acids.

In some embodiments, the ligand binding domain comprises a VH domain and a VL domain, wherein a polynucleotide encoding the VH domain is 3′ of a polynucleotide encoding the VH domain; the spacer comprises an amino acid sequence having at least 95% identity to the amino acid sequence of any one of SEQ ID NO:16; and the spacer comprises less than 120 consecutive amino acids.

In some embodiments, the transmembrane domain comprises a CD28 transmembrane domain. In some embodiments, the CD28 transmembrane domain comprises an amino acid sequence having at least 95% identity to the amino acid sequence of SEQ ID NO:20.

In some embodiments, the intracellular signalling domain comprises a costimulatory domain selected from the group consisting of CD27, CD28, 4-1BB, OX-40, CD30, CD40, PD-1, ICOS, LFA-1, CD2, CD7, NKG2C, and B7-H3, in combination with a CD3-zeta domain or functional portion thereof. In some embodiments, the intracellular signalling domain comprises a 4-1BB costimulatory domain. In some embodiments, the 4-1BB costimulatory domain is encoded by a nucleotide sequence having at least 95% identity to the nucleotide sequence of SEQ ID NO:22.

In some embodiments, the CD3-zeta domain or functional portion thereof is encoded by a nucleotide sequence having at least 95% identity to the nucleotide sequence of SEQ ID NO:23.

Some embodiments also include a polynucleotide encoding a cell surface selectable marker. In some embodiments, the cell surface selectable marker comprises a truncated EGFR polypeptide (EGFRt).

Some embodiments also include a ribosome skip sequence. In some embodiments, the ribosome skip sequence is selected from the group consisting of a P2A sequence, a T2A sequence, an E2A sequence, and an F2A sequence.

In some embodiments, a polynucleotide encoding the CAR is operably linked to an EF1α promoter.

In some embodiments, a polynucleotide encoding the CAR is operably linked to an inducible promoter.

Some embodiments also include a polynucleotide encoding a suicide gene system. In some embodiments, the suicide gene system is selected from a herpes simplex virus thymidine kinase/ganciclovir (HSVTK/GCV) suicide gene system, or an inducible caspase suicide gene system.

Some embodiments of the methods and compositions provided herein include a polypeptide encoded by any one of the foregoing nucleic acids.

Some embodiments of the methods and compositions provided herein include a vector comprising any one of the foregoing nucleic acids. In some embodiments, the vector is selected from the group consisting of a viral vector, a transposon vector, an integrase vector, and an mRNA vector. In some embodiments, the viral vector is selected from the group consisting of a lentiviral vector, a foamy viral vector, a retroviral vector, and a gamma retroviral vector. In some embodiments, the viral vector is a lentiviral vector.

Some embodiments of the methods and compositions provided herein include a host cell comprising any one of the foregoing nucleic acids.

In some embodiments, the host cell is a CD4+ T-cell or a CD8+ T-cell.

In some embodiments, the host cell is a precursor T-cell, or a hematopoietic stem cell.

In some embodiments, the host cell is a CD8+ cytotoxic T-cell selected from the group consisting of a naïve CD8+ T-cell, a CD8+ memory T-cell, a central memory CD8+ T-cell, a regulatory CD8+ T-cell, an IPS derived CD8+ T-cell, an effector memory CD8+ T-cell, and a bulk CD8+ T-cell.

In some embodiments, the host cell is a CD4+ T helper cell selected from the group consisting of a naïve CD4+ T-cell, a CD4+ memory T-cell, a central memory CD4+ T-cell, a regulatory CD4+ T-cell, an IPS derived CD4+ T-cell, an effector memory CD4+ T-cell, and a bulk CD4+ T-cell.

Some embodiments include any one of the foregoing host cells for use as a medicament.

Some embodiments of the methods and compositions provided herein include a pharmaceutical composition comprising any one of the foregoing host cells, and a pharmaceutically acceptable excipient.

Some embodiments include any one of the foregoing host cells for use in treating, ameliorating or inhibiting a cancer in a subject.

Some embodiments include any one of the foregoing host cells for use in treating, ameliorating or inhibiting a cancer in a subject in combination with a donor cell comprising a short isoform of a CD33 protein (CD33m) and lacking a long isoform of a CD33 protein (CD33M).

In some embodiments, the host cell and/or the donor cell is allogeneic to the subject.

In some embodiments, the host cell and/or the donor cell is autologous to the subject.

In some embodiments, the short isoform of a CD33 protein lacks an epitope for specific binding of the CAR.

In some embodiments, the short isoform of a CD33 protein lacks an IgV domain.

In some embodiments, the donor cell is a hematopoietic stem cell.

In some embodiments, the donor cell is administered prior to administration of the host cell.

In some embodiments, the donor cell and the host cell are administered concurrently.

In some embodiments, the cancer comprises a CD33 expressing cancer cell.

In some embodiments, the cancer comprises a leukemia. In some embodiments, the cancer is selected from an acute myeloid leukemia (AML), a chronic myelogenous leukemia (CML), and a chronic granulocytic leukemia (CGL).

In some embodiments, the subject is mammalian. In some embodiments, the subject is human.

In some embodiments, the donor cell is obtained by genetically modifying a substrate cell to remove expression of the long isoform of a CD33 protein (CD33M) in the substrate cell, thereby obtaining the donor cell. In some embodiments, genetically modifying a substrate cell comprises use of a system selected from the group consisting of a CRISPR-CAS system, a zinc finger nuclease system, and a transcription activator-like effector nuclease (TALEN) system.

Some embodiments of the methods and compositions provided herein include a method of treating, inhibiting or ameliorating a cancer in a subject, the method comprising: administering any one of the foregoing host cells to the subject.

Some embodiments also include administering a donor cell comprising a short isoform of a CD33 protein (CD33m) and lacking a long isoform of a CD33 protein (CD33M).

In some embodiments, the host cell and/or the donor cell is allogeneic to the subject.

In some embodiments, the host cell and/or the donor cell is autologous to the subject.

In some embodiments, the short isoform of a CD33 protein lacks an epitope for specific binding of the CAR.

In some embodiments, the short isoform of a CD33 protein lacks an IgV domain.

In some embodiments, the donor cell is a hematopoietic stem cell.

In some embodiments, the donor cell is administered prior to administration of the host cell.

In some embodiments, the donor cell and the host cell are administered concurrently.

In some embodiments, the cancer comprises a CD33 expressing cancer cell.

In some embodiments, the cancer comprises a leukemia. In some embodiments, the cancer is selected from an acute myeloid leukemia (AML), a chronic myelogenous leukemia (CML), and a chronic granulocytic leukemia (CGL).

In some embodiments, the subject is mammalian. In some embodiments, the subject is human.

Some embodiments also include genetically modifying a substrate cell to remove expression of the long isoform of a CD33 protein (CD33M) in the substrate cell, thereby obtaining the donor cell. In some embodiments, genetically modifying a substrate cell comprises use of a system selected from the group consisting of a CRISPR-CAS system, a zinc finger nuclease system, and a transcription activator-like effector nuclease (TALEN) system.

Some embodiments of the methods and compositions provided herein include a method of preparing a population of host cells, comprising: introducing any one of the foregoing nucleic acids into an isolated cell; and culturing the cell in the presence of an agent selected from an anti-CD3, an anti-CD28, and a cytokine, thereby obtaining the population of host cells.

In some embodiments, the host cell is a precursor T-cell, or a hematopoietic stem cell.

In some embodiments, the host cell is a CD8+ cytotoxic T-cell selected from the group consisting of a naïve CD8+ T-cell, a CD8+ memory T-cell, a central memory CD8+ T-cell, a regulatory CD8+ T-cell, an IPS derived CD8+ T-cell, an effector memory CD8+ T-cell, and a bulk CD8+ T-cell.

In some embodiments, the host cell is a CD4+ T helper cell selected from the group consisting of a naïve CD4+ T-cell, a CD4+ memory T-cell, a central memory CD4+ T-cell, a regulatory CD4+ T-cell, an IPS derived CD4+ T-cell, an effector memory CD4+ T-cell, and a bulk CD4+ T-cell.

In some embodiments, the cytokine is selected from the group consisting of IL2, IL7, IL12, and IL15.

### BRIEF DESCRIPTION OF THE DRAWINGS

FIG. 1 is a diagram showing the structure of polynucleotides encoding the CD33M (CD33 Iso-L protein or long isoform) and the CD33m (CD33 Iso-S protein or short isoform).

FIG. 2 is a diagram outlining an embodiment of a therapy plan for a leukemia patient. As shown, donor cells are provided that are homozygous for expression of CD33m (CD33 Iso-S protein or short isoform). The cells are transplanted into a patient. Following engraftment, the patient is then administered donor CAR T cells that are specific for CD33M (CD33 Iso-L protein or long isoform).

FIG. 3 is a diagram outlining an embodiment of a therapy plan for a leukemia patient. As shown, donor cells are provided from a related or a non-related donor of the patient. The cells are heterozygous or homozygous for expression of CD33M (CD33 Iso-L protein or long isoform). The cells are edited using a zinc finger endonuclease, TALEN, CRISPR-CAS system or MegaTalen for expression of CD33m (CD33 Iso-S protein or short isoform). The cells are transplanted into a patient. Following engraftment, the patient is then administered donor CAR T cells that are specific for CD33M (CD33 Iso-L protein or long isoform).

FIG. 4 is a diagram outlining an embodiment of a therapy plan for a leukemia patient. As shown, donor cells are provided from the patient. The cells are heterozygous or homozygous for expression of CD33M (CD33 Iso-L protein or long isoform). The cells are edited using a zinc finger endonuclease, TALEN, CRISPR-CAS system or MegaTalen for expression of CD33m (CD33 Iso-S protein or short isoform). The cells are transplanted into a patient. Following engraftment, the patient is then administered donor CAR T cells that are specific for CD33M (CD33 Iso-L protein or long isoform).

FIG. 5 is a schematic of a polynucleotide encoding a CD33 targeting CAR, which is specific for CD33M (CD33 Iso-L protein or long isoform).

FIG. 6 depicts a flow cytometry analysis of either CD4+ T cells or CD8+ T cells transduced with various polynucleotides encoding CD33 targeting CARs.

FIG. 7 depicts graphs of specific lysis of CD33+ target cells incubated at various ratios with effector CD8+ T cells transduced with various polynucleotides encoding CD33 targeting CARs.

FIG. 8 depicts graphs of cytokine production (IL-2, IFN-γ, and TNF-α) from CD4+ T cells containing various CD33 targeting CARs and, which were incubated with CD33+ target cells.

FIG. 9 depicts a time-line for therapy utilizing a xenograft model of AML with T cells containing CD33 targeting CARs.

FIG. 10 depicts a Kaplan-Meier survival curve for mice, which received T cells containing CD33 targeting CARs.

FIG. 11 depicts graphs of total flux (photons/sec) as a measure of tumor burden (y-axis) over time (days post-tumor inoculation, x-axis), for mice, which received mock cells, or T cells containing CD33 targeting CARs.

FIG. 12 depicts a series of photographs showing bioluminescent images of mice prior to and following administration of CD33CARs until the end of the study.

### DETAILED DESCRIPTION

Some embodiments of the methods and compositions provided herein concern chimeric antigen receptors (CAR)s, which specifically bind to an epitope of CD33, such as an epitope encoded by exon 2 of CD33. Some embodiments include the use of such CARs in effective and safe therapies for myeloid leukemias, such as acute myeloid leukemia or chronic myeloid leukemia.

The use of CAR T-cell therapy has been previously described for treating cancers as the CARs may be designed to specifically bind to proteins or tumor signature proteins in order to elicit the immune system to destroy cancerous cells. Several CAR T cell therapies have recently been approved by the Food and Drug Administration (FDA) for the treatment of acute lymphoblastic leukemia, a type of large B-cell lymphoma. CAR T cell targets that have been previously described for treating leukemia include CD19.

CD19 is a B-cell surface protein expressed throughout B-cell development; therefore, it is expressed on nearly all B-cell malignancies, including chronic lymphocytic leukemia (CLL), ALL, and many non-Hodgkin lymphomas. This near-universal expression and specificity for a single cell lineage has made CD19 an attractive target for CAR-modified T-cell therapies. Unfortunately, CD19 is also found in normal cells such as non-malignant B cells. Ablation of CD19 expressing cells may be tolerated by patients undergoing CD19 directed CAR T-cell therapy as the patient may still rely on antibody producing CD19− plasma cells and a patient may also simultaneously be treated with IVIG immunoglobulin replacement therapy.

CD33 is another target for CAR T cell therapy for myleogenous leukemia therapies. CD33 is a myeloid cell surface antigen, which is expressed in a high percentage of myelogenous leukemias and has been the target for antibody-based therapeutics and CAR T cell therapy. CD33 is expressed by non-malignant myeloid derived cells that have roles for host protection from infectious organisms, which complicates the use of CD33-directed CAR T cell therapies. Unlike CD19-expressing B cells, the replacement of myeloid cell protective function cannot be replaced by alternate CD33-nonexpressing cell sources. In addition, CD33 is expressed in myeloid progenitors and targeting CD33 by CAR T cell therapy risks causing pancytopenias or marrow ablation. Accordingly, the potential side-effect of a CAR T-cell therapy, which targets CD33, is the death of B cells en masse leading to B cell aplasia.

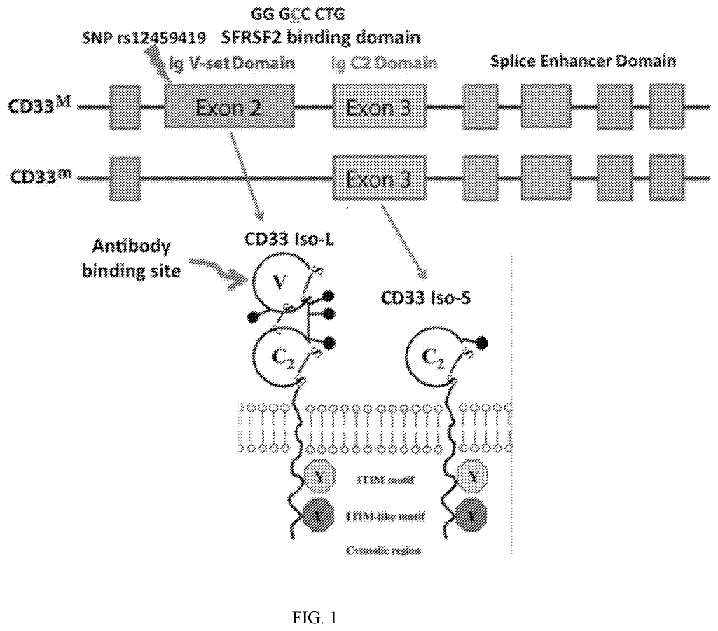

Some embodiments described herein address these obstacles. A single nucleotide polymorphism (SNP) has been identified in the exon 2 coding region of the CD33 gene (SNP rs12459419). This SNP is located in the spliceosome recognition site, which affects the amino acid composition of the extracellular domain of CD33. Variation in this SNP regulates inclusion or exclusion of exon 2, which encodes the extracellular IgV domain of this protein, leading to expression of the long form of CD 33 (CD33 Iso-L also referred to as CD33M) or short isoforms of CD33 (CD33 Iso-S also referred to herein as CD33m) (see FIG. 1). In some embodiments, a long isoform of a CD33 protein includes an extracellular IgG domain, and an IgV domain; and a short isoform of a CD33 protein includes an extracellular IgG domain and lacks an IgV domain.

Patients homozygous for the long isotope of CD33 are also referred to as CD33M/M and patients homozygous for the short isotope are also referred to as CD33m/m. Patients who are heterozygotes are referred to as CD33M/m. In this model, patients with SNP rs12459419 CC (CD33M/M) genotype lead to retention of exon 2 (CD33 Iso-L (CD33M); whereas those with SNP rs12459419 TT (CD33m/m) genotype lead to exclusion of exon 2 (CD33 Iso-S(CD33m)). The majority of antiCD33 antibodies, including the antibody used in the ADC Myelotarg (known commercially as the antibody portion of Gemtuzumab Ozogamicin (Mylotarg®), which is IgG4κ antibody hP67.6), recognize an epitope, which is present exclusively in the long isoform of CD33 (CD33 Iso-L (CD33M)). As shown in FIG. 1, retaining exon 2 leads to an isoform that retains the IgV domain (CD33M). Deletion of exon 2 leads to the CD33m form, which only has the IgC2 domain (CD33m).

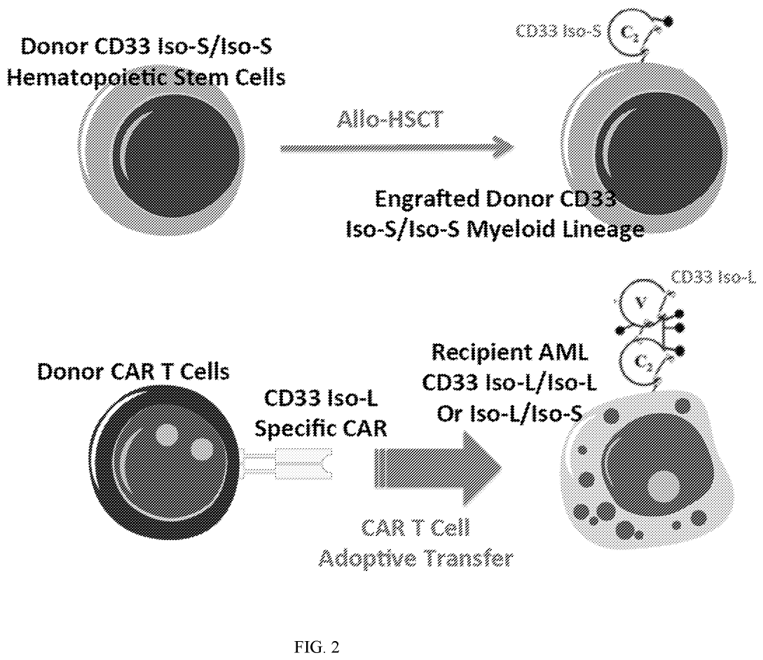

In order to obviate the limitations of therapies targeting the ubiquitously expressed CD33 marker, alternative methods have been developed, as described herein, which includes the use of CD33M (CD33 Iso-L protein or long isoform) specific CAR T cell adoptive immunotherapy that are derived from donors that are CD33M/m homozygous following allogeneic transplant of patients whose leukemia expresses CD33M. In this setting, after donor hematopoietic engraftment, CD33M expressed by myeloid leukemias functions as a tumor specific cell surface antigen for CAR T cell targeting. An example embodiment is shown in FIG. 2 in which donor hematopoietic stem cells from an individual are obtained and through allogeneic hematopoietic stem cell transplantation of cells that express CD33m (CD33 Iso-S protein or short isoform) said cells are engrafted into an individual that is homozygous for CD33M/M or heterozygous CD33M/m individuals that expresses the CD33M isoform. The patient is subsequently administered a CAR T cell therapy against the long isoform of CD33 (CD33M).

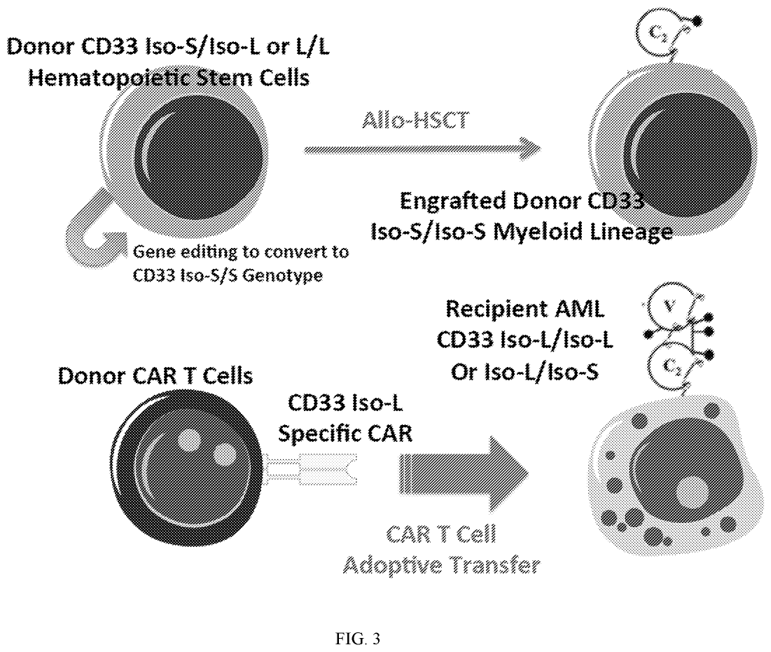

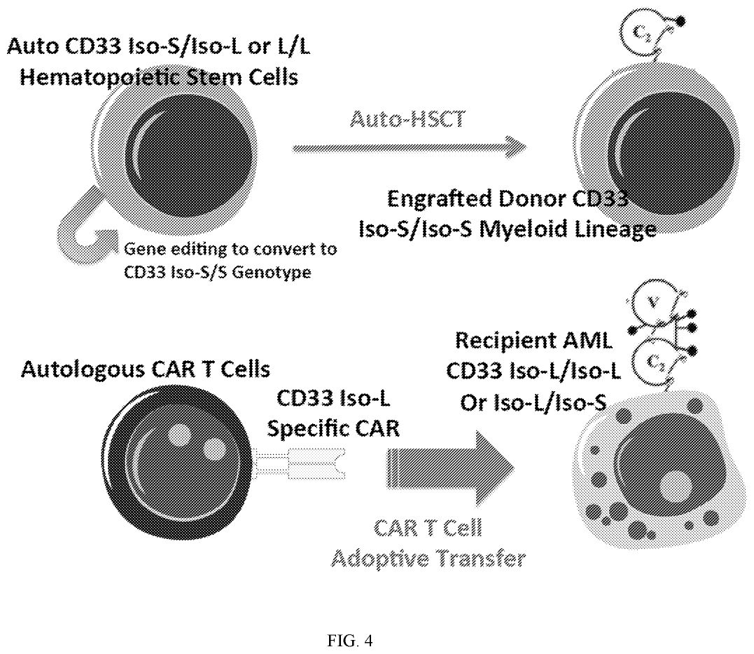

Some embodiments described herein include genetic engineering of donor hematopoietic stem cells to convert donor SNP rs12459419 CC (CD33M/M) or CT genotype (CD33M/m) to TT genotype (CD33m/m), thus creating a genotype and phenotype mismatch between the donor and recipient. This gene editing can provide donor cells with homozygous CD33 Iso-S(CD33m) and host (leukemic cells) with CD33 Iso-L phenotype (CD33M). There are a variety of gene editing approaches that use a targeted nuclease, such as zinc finger, TALEN, CRISPR, or mega Talen moieties. These approaches can be used to knock-out expression of a CD33 Iso-L (CD33M) allele in donors that are heterozygous, and/or convert Iso-S alleles (CD33m) to Iso-L alleles (CD33M) by homologous recombination of donor DNA sequences encoding the Iso-S(CD33m) specific sequence. In some embodiments, the cells may be from family donors or unrelated donors (FIG. 3). In some embodiments, cells may also be taken for gene editing from the patient in need of the therapy, for instance, hematopoietic stem cells are taken from the patient and used in a gene editing method to express CD33m (autologous HSCT, FIG. 4). Following transplant and upon donor T cell engraftment T, Iso-S (CD33m/m) homozygous T cells are harvested and CD33 Iso-L (CD33M) specific CAR T cells are manufactured and reinfused into the patient so as to target residual leukemia. Alternately, donor T cells can be modified to both express the CD33 Iso-L (CD33M) CAR T cells, as T cells do not express CD33. (See FIG. 3 and FIG. 4).

In some embodiments, a leukemia patient's Iso-L (CD33M) HSC's are purged of contaminating leukemic cells and are converted to Iso-s by gene editing at the CD33 locus for autologous transplant followed by autologous or third party CD33M CAR T cell immunotherapy.

Thus, some embodiments provided herein relate to a myeloid leukemia-specific CAR T cell immunotherapy, wherein Iso-L CD33 (CD33M) is targeted in the context of normal hematopoiesis of myeloid cells that are exclusively Iso-S CD33.

### Definitions

Unless defined otherwise, all technical and scientific terms used herein have the same meaning as commonly understood by one of ordinary skill in the art to which the invention pertains.

As used herein, “a” or “an” may mean one or more than one.

“About” as used herein when referring to a measurable value is meant to encompass variations of 20% or ±10%, more preferably +5%, even more preferably +1%, and still more preferably +0.1% from the specified value.

As used herein, “nucleic acid” or “nucleic acid molecule” have their plain and ordinary meaning in view of the whole specification and may to refer to, for example, polynucleotides, such as deoxyribonucleic acid (DNA) or ribonucleic acid (RNA), oligonucleotides, fragments generated by the polymerase chain reaction (PCR), or fragments generated by any of ligation, scission, endonuclease action, or exonuclease action. Nucleic acid molecules can be composed of monomers that are naturally-occurring nucleotides (such as DNA or RNA), or analogs of naturally-occurring nucleotides (e.g., enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, or azido groups, or sugars can be functionalized as ethers or esters. Moreover, the entire sugar moiety can be replaced with sterically and electronically similar structures, such as aza-sugars or carbocyclic sugar analogs. Examples of modifications in a base moiety include alkylated purines or pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, or phosphoramidate, and the like. The term “nucleic acid molecule” also includes so-called “peptide nucleic acids,” which comprise naturally-occurring or modified nucleic acid bases attached to a polyamide backbone. Nucleic acids can be either single stranded or double stranded. In some embodiments, a nucleic acid sequence encoding a fusion protein is provided. In some embodiments, the nucleic acid encoding the chimeric antigen receptor specific for CD33M (CD33 Iso-L protein or long isoform) is RNA or DNA.

As used herein, “coding for” or “encoding” has its plain and ordinary meaning when read in light of the specification, and includes, for example, the property of specific sequences of nucleotides in a polynucleotide, such as a gene, a cDNA, or an mRNA, to serve as templates for synthesis of other macromolecules such as a defined sequence of amino acids. Thus, a gene codes for a protein if transcription and translation of mRNA corresponding to that gene produces the protein in a cell or other biological system.

As used herein, “chimeric antigen receptor” has its plain and ordinary meaning when read in light of the specification, and may include but is not limited to, for example, a synthetically designed receptor comprising a ligand binding domain of an antibody or other protein sequence that binds to a molecule associated with a disease or disorder and is, preferably, linked via a spacer domain to one or more intracellular signaling domains of a cell, such as a T cell, or other receptors, such as one or more costimulatory domains. Chimeric receptor can also be referred to as artificial cell receptors or T cell receptors, chimeric cell receptors or T cell receptors, chimeric immunoreceptors, or chimeric antigen receptors (CARs). These receptors can be used to graft the specificity of a monoclonal antibody or binding fragment thereof onto a cell, preferably a T-cell, with transfer of their coding sequence facilitated by viral vectors, such as a retroviral vector or a lentiviral vector. CARs can be, in some instances, genetically engineered T cell receptors designed to redirect T cells to target cells that express specific cell-surface antigens. T cells can be removed from a subject and modified so that they can express receptors that can be specific for an antigen by a process called adoptive cell transfer. The T cells are reintroduced into the patient where they can then recognize and target an antigen. CARs are also engineered receptors that can graft an arbitrary specificity onto an immune receptor cell. The term chimeric antigen receptors or “CARs” are considered by some investigators to include the antibody or antibody fragment, preferably an antigen binding fragment of an antibody, the spacer, signaling domain, and transmembrane region. Due to the surprising effects of modifying the different components or domains of the CAR described herein, such as the epitope binding region (for example, antibody fragment, scFv, or portion thereof), spacer, transmembrane domain, and/or signaling domain), the components of the CAR are frequently distinguished throughout this disclosure in terms of independent elements. The variation of the different elements of the CAR can, for example, lead to a desired binding affinity, such as a stronger binding affinity for a specific epitope or antigen.

The CARs graft the specificity of a monoclonal antibody or binding fragment thereof or scFv onto a T cell, with the transfer of their coding sequence facilitated by vectors. In order to use CARs as a therapy for a subject in need, a technique called adoptive cell transfer is used in which T cells are removed from a subject and modified so that they can express the CARs that are specific for an antigen. The T cells, which can then recognize and target an antigen, are reintroduced into the patient.

In some embodiments, the transmembrane domain is a region of a membrane-spanning protein that is hydrophobic that can reside in the bilayer of a cell to anchor a protein that is embedded to the biological membrane. Without being limiting, the topology of the transmembrane domain can be a transmembrane alpha helix. In some alternatives of the chimeric antigen receptor, the chimeric antigen receptor comprises a sequence encoding a transmembrane domain. In some alternatives, the transmembrane domain comprises a CD28 transmembrane sequence or a fragment thereof that is a length of 10, 11, 12, 13, 14, 15, 16, 17, 18, 19, 20, 21, 22, 23, 24, 25, 26, 27, or 28 amino acids or a length within a range defined by any two of the aforementioned lengths. In some alternatives, the CD28 transmembrane sequence or fragment thereof comprises 28 amino acids in length.

In some embodiments, the signaling domains, such as primary signaling domains or costimulatory domains, include an intracellular or cytoplasmic domain of a protein or a receptor protein that interacts with components within the interior of the cells and is capable of or configured to relay or participate in the relaying of a signal. Such interactions in some aspects can occur through the intracellular domain communicating via specific protein-protein or protein-ligand interactions with an effector molecule or an effector protein, which in turn can send the signal along a signal chain to its destination. In some embodiments, the signaling domain includes one or more co-stimulatory domains. In some aspects, the one or more costimulatory domains include a signaling moiety that provides a T-cell with a signal, which, in addition to the primary signal provided by for instance the CD3 zeta chain of the TCR/CD3 complex, enhances a response such as a T-cell effector response, such as, for example, an immune response, activation, proliferation, differentiation, cytokine secretion, cytolytic activity, perforin or granzyme activity or any combination thereof. In some embodiments, the intracellular signaling domain or the co-stimulatory domain can include all or a portion of CD27, CD28, 4-1BB, OX40, CD30, CD40, ICOS, lymphocyte function-associated antigen-1 (LFA-1), CD2, CD7, LIGHT, NKG2C, or B7-H3, or a ligand that specifically binds with CD83 or any combination thereof.

As used herein, an “antibody” has its plain and ordinary meaning when read in light of the specification, and includes, for example, a large Y-shape protein produced by plasma cells that is used by the immune system to identify and neutralize foreign objects such as bacteria and viruses. The antibody protein can comprise four polypeptide chains; two identical heavy chains and two identical light chains connected by disulfide bonds. Each chain is composed of structural domains called immunoglobulin domains. These domains can contain about 70, 80, 90, 100, 110, 120, 130, 140, or 150 amino acids or any number of amino acids in between in a range defined by any two of these values and are classified into different categories according to their size and function. In some embodiments, the ligand binding domain comprises an antibody or binding fragment thereof or scFv, a receptor ligand or mutants thereof, peptide, and/or polypeptide affinity molecule or binding partner. In some embodiments, the ligand binding domain is an antibody fragment, desirably, a binding portion thereof. In some embodiments, the antibody fragment or binding portion thereof present on a CAR is specific for a ligand on a B-cell. In some embodiments, the antibody fragment or binding portion thereof present on a CAR or TcR is specific for a ligand. In some embodiments, the antibody fragment or binding portion thereof present on a CAR is specific for CD33M (CD33 Iso-L protein or long isoform). In some embodiments, the ligand binding domain is an antibody fragment or a binding portion thereof, such as a single chain variable fragment (scFv). In some embodiments, the antibody fragment or binding portion thereof present on a CAR comprises one or more domains from a humanized antibody, or binding portion thereof.

As used herein, a “single chain variable fragment” or “scFv” has its plain and ordinary meaning when read in light of the specification, and includes, for example, a fusion protein of the variable regions of the heavy (VH) and light chains (VL) of immunoglobulins, connected with a short linker peptide of ten to about 25 amino acids. In some embodiments, a CAR is provided, wherein the CAR comprises a ScFv specific for CD33M (CD33 Iso-L protein or long isoform). In some such embodiments, the CAR is capable of specifically binding to a polypeptide and/or epitope encoded by exon 2 of the CD33 gene. Example sequences of VH and VL domains for scFvs useful in embodiments provided herein are listed in TABLE 2.

The strength of binding of a ligand is referred to as the binding affinity and can be determined by direct interactions and solvent effects. A ligand can be bound by a “ligand binding domain.” A ligand binding domain, for example, can refer to a conserved sequence in a structure that can bind a specific ligand or a specific epitope on a protein. The ligand binding domain or ligand binding portion can comprise an antibody or binding fragment thereof or scFv, a receptor ligand or mutants thereof, peptide, and/or polypeptide affinity molecule or binding partner. Without being limiting, a ligand binding domain can be a specific protein domain or an epitope on a protein that is specific for a ligand or ligands.

Some embodiments include a spacer. In some alternatives, the peptide spacer is 15 amino acids or less but not less than 1 or 2 amino acids. In some embodiments, the spacer is a polypeptide chain. In some aspects, the polypeptide chain may range in length, such as from 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101,102, 103,104, 105, 106,107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239 or 240 amino acids or a length within a range defined by any two of the aforementioned lengths. A spacer can comprise any 20 amino acids, for example, in any order to create a desirable length of polypeptide chain in a chimeric antigen receptor, which includes the amino acids arginine, histidine, lysine, aspartic acid, glutamic acid, serine, threonine, asparagine, glutamine, cysteine, glycine, proline, alanine, valine, isoleucine, methionine, phenylalanine, tyrosine or tryptophan. A spacer sequence can be a linker between the scFv (or ligand binding domain) and the transmembrane domain of the chimeric antigen receptor. In some alternatives of the chimeric antigen receptor, the chimeric antigen receptor further comprises a sequence encoding a spacer. In some alternatives the spacer comprises a sequence with a length of 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239 or 240 amino acids or a length within a range defined by any two of the aforementioned lengths. In some alternatives, the spacer resides between the scFv and the transmembrane region of the chimeric antigen receptor. In some alternatives, the spacer resides between the ligand binding domain of the chimeric antigen receptor and the transmembrane region of the chimeric antigen receptor.

A spacer may also be customized, selected, or optimized for a desired length so as to improve or modulate binding of scFv domain to the target cell, which may increase or provide the desired amount of cytotoxic efficacy. In some alternatives, the linker or spacer between the scFv domain or ligand binding domain and the transmembrane can be 25 to 55 amino acids in length (e.g., at least, equal to 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, or 55 amino acids or a length within a range defined by any two of the aforementioned lengths).

In some alternatives, the spacer comprises a hinge region of a human antibody. In some alternatives, the spacer comprises an IgG4 hinge. In some alternatives, the IgG4 hinge region is a modified IgG4 hinge. A “modified IgG4 hinge” as described herein can refer to a hinge region that can have at least 90%, 91%, 92%, 93%, 94% 95%, 96%, 97%, 98%, 99%, or 100% sequence identity or a sequence identity within a range defined by any two of the aforementioned percentages, with a hinge region amino acid sequence as set forth in a spacer, such as a short spacer listed in TABLE 2.

In some alternatives, the CAR comprises an S spacer, M spacer or an L spacer. Example sequences are listed in TABLE 2.

As used herein, a “de-immunized spacer” has its plain and ordinary meaning when read in light of the specification, and includes, for example, a spacer that induces little to no immune response or a diminished or reduced immune response from a patient. In some embodiments, the chimeric antigen receptor comprises a spacer, wherein the spacer does not induce an immune response in a subject, such as a human. It is important that the spacer does not induce an immune response or induces a reduced or diminished or low immune response in a subject, such as a human, in order to prevent or reduce the ability of the immune system to attack the chimeric antigen receptor.

As used herein, a “transmembrane domain” is a region of a protein that is hydrophobic that can reside in the bilayer of a cell to anchor a protein that is embedded to the biological membrane. Without being limiting, the topology of the transmembrane domain can be a transmembrane alpha helix. In some alternatives of the method of making genetically modified T-cells, which have a chimeric antigen receptor, the vector comprises a sequence encoding a transmembrane domain. In some alternatives of the method, the transmembrane domain comprises a CD28 transmembrane sequence or a fragment thereof that is a length of 10, 11, 12, 13, 14, 15, 16, 17, 18, 19, 20, 21, 22, 23, 24, 25, 26, 27, or 28 amino acids or a length within a range defined by any two of the aforementioned lengths. In some embodiments, the CD28 transmembrane sequence or fragment thereof comprises at least 28 amino acids in length.

As used herein, “signaling domain” is a domain on a chimeric antigen receptor that can promote cytokine release, in vivo T cell survival and tumor elimination. In some alternatives herein, a signaling domain comprises CD28, 4-1BB or CD3-zeta cytoplasmic domains or any combination thereof.

As used herein, a “ribosome skip sequence” has its plain and ordinary meaning when read in light of the specification, and includes, for example, a sequence that during translation, forces the ribosome to “skip” the ribosome skip sequence and translate the region after the ribosome skip sequence without formation of a peptide bond. Several viruses, for example, have ribosome skip sequences that allow sequential translation of several proteins on a single nucleic acid without having the proteins linked via a peptide bond. As described herein, this is the “linker” sequence. In some alternatives of the nucleic acids provided herein, the nucleic acids comprise a ribosome skip sequence between the sequence for the chimeric antigen receptor and the sequence of the marker protein, such that the proteins are co-expressed and not linked by a peptide bond. In some embodiments, the ribosome skip sequence is a P2A, T2A, E2A or F2A sequence.

As used herein, a “marker sequence,” has its plain and ordinary meaning when read in light of the specification, and includes, for example, a protein that is used for selecting or tracking a protein or cell that has a protein of interest. In the alternatives described herein, the fusion protein provided can comprise a marker sequence that can be selected in experiments, such as flow cytometry. In some embodiments, the marker is the protein Her2tG or EGFRt.

As used herein, “signal sequence” for secretion, can also be referred to as a “signal peptide.” The signal peptide can be used for secretion efficiency and in some systems, it is recognized by a signal recognition particle, which halts translation and directs the signal sequence to an SRP receptor for secretion. In some alternatives of the CARs provided herein, the CARs further comprise a signal sequence. In some embodiments, of the nucleic acid encoding a CAR, the nucleic acid comprises a sequence encoding a signal sequence. In some embodiments, the signal sequence is for targeting a protein to a cell membrane following translation of the protein.

As used herein, “TamR-tf”, also designated “HEA3” has its plain and ordinary meaning when read in light of the specification, and includes, for example, a chimeric transcription factor composed of human subunits including the N-terminal DNA binding domain of Hepatocyte Nuclear Factor 1-alpha (HNF-1α) fused in frame to the mutant tamoxifen-specific ligand binding domain of the estrogen receptor ligand binding domain (ER-LBD), that is in turn fused to the p65 activation domain of NF-κB (p65). In some embodiments, the polynucleotide comprises a synthetic transcriptional regulator, which, in the presence of tamoxifen, binds a synthetic promoter upstream of a transgene to induce expression. The mutant tamoxifen-specific ligand binding domain of the estrogen receptor ligand binding domain (ER-LBD) is found at amino acids 282-595 of the TamR-tf and has a mutation at position 521. Further changes can be made to the transcriptional activator to increase the properties of the transcription factor including, without limitation, altering one or more amino acids in the estrogen receptor ligand binding domain to increase the affinity of the factor for estrogen analogs and altering one or more amino acids in the p65 transactivating domain.

In the absence of tamoxifen, TamR-tf is excluded from the nucleus by binding of cytosolic heat-shock protein 90 (HSP90) to the tamoxifen binding active site and transgene expression is in the “OFF” state. Nanomolar concentrations of cytosolic tamoxifen actively out competes HSP90 for ER-LBD binding, resulting in TamR-tf translocation to the nucleus. Upon nuclear translocation, TamR-tf is readily available to bind its restricted synthetic promoter (e.g. 7×HBD/EF1αp). In the presence of tamoxifen, binding of TamR-tf to 7×HBD/EF1αp promoter induces the “ON” state of transgene expression. In some embodiments, this transcriptional regulator can be modified to provide for a varying level of control of transgene expression. Amino acid substitutions in the LBD of TamR-tf permit selective responsiveness to tamoxifen and its metabolites, where 4-hydroxy tamoxifen (4-OHT) is the most pharmacologically active metabolite in regards to TamR-tf activity, while lacking interaction with endogenous estrogen. In some embodiments, a nucleic acid encoding a chimeric antigen receptor is provided, wherein the nucleic acid comprises a promoter regulated by a drug. In some embodiments, the drug includes tamoxifen, its metabolites, analogs, or pharmaceutically acceptable salts or hydrates or solvates thereof.

Tamoxifen, CAS RN: 10540-29-1, is also known as 2-(4-((1Z)-1,2-diphenyl-1-butenyl)phenoxy)-N,N-dimethyl-ethanamine, or (Z)-2-(para-(1,2-Diphenyl-1-butenyl)phenoxy)-N,N-dimethylamine (IUPAC), and has a molecular formula of C26H29NO, M.W. 371.52. Tamoxifen is a Selective Estrogen Receptor Modulator with tissue-specific activities. Tamoxifen acts as an anti-estrogen (inhibiting agent) agent in the mammary tissue, but as an estrogen (stimulating agent) in cholesterol metabolism, bone density, and cell proliferation in the endometrium. Tamoxifen is frequently administered orally as a pharmaceutically acceptable salt. For example, Tamoxifen citrate (RN 54965-24-1, M.W. 563.643) is indicated for treatment of metastatic breast cancer, and as an adjuvant for the treatment of breast cancer in women following mastectomy axillary dissection, and breast irradiation.

Metabolites of tamoxifen in rat, mouse and human breast cancer patients, including major metabolites N-desmethyltamoxifen (RN 31750-48-8, M.W. 357.494) and 4-hydroxytamoxifen (4-OHT) (RN 68392-35-8, M.W. 387.52, Afimoxifene), are disclosed in Robinson et al., Metabolites, pharmacodynamics, and pharmacokinetics of tamoxifen in rats and mice compared to the breast cancer patient. Drug Metab Dispos January 1991 19:36-43, which is incorporated by reference herein in its entirety. Additional cytochrome P-450 metabolites are disclosed in Crewe et al., 2002, including cis-4-hydroxytamoxifen (RN 174592, M.W. 387.52; Afimoxifene, E-isomer), and 4′-hydroxytamoxifen ((Z)-4-(1-(4-(2-(dimethylamino)ethoxy)phenyl)-1-phenylbut-1-en-2-yl)phenol). See Crewe et al., 2002, Metabolism of Tamoxifen by recombinant human cytochrome P-450 enzymes: Formation of the 4-hydroxy, 4′-hydroxy and N-desmethyl metabolites and isomerization of trans-4-hydroxytamoxifen, Drug Metab Dispos, 30(8): 869-874, FIG. 1, which is expressly incorporated herein by reference in its entirety.

As used herein, “suicide gene therapy,” “suicide genes” and “suicide gene systems” have their plain and ordinary meaning when read in light of the specification, and includes, for example, methods to destroy a cell through apoptosis, which requires a suicide gene that will cause a cell to kill itself by apoptosis. Due to safety concerns for the patients in need of using genetically modified immune cells for treatment or modification of a tumor environment, strategies are being developed in order to prevent or abate adverse events. Adverse effects of incorporation of genetically modified immune cells into a subject for a pretreatment step can include “cytokine storms,” which is a cytokine release syndrome, wherein the infused T-cells release cytokines into the bloodstream, which can lead to dangerously high fevers, as well as, a precipitous drop in blood pressure. Control of the system by tamoxifen, as previously described, may also be used when there is indication of a cytokine storm, such as a fever.

As used herein, “vector” or “construct” has its plain and ordinary meaning when read in light of the specification, and includes, for example, a nucleic acid used to introduce heterologous nucleic acids into a cell that has regulatory elements to provide expression of the heterologous nucleic acids in the cell. Vectors include but are not limited to plasmid, minicircles, yeast, viral genomes, lentiviral vector, foamy viral vector, retroviral vector or gammaretroviral vector. The vector may be DNA or RNA, such as mRNA.

As used herein, “transposon gene cassettes” or transposons refers to a genetic element that contains a gene (promoter that drives expression of a primary transcript), flanked by recombinase recognition sites (for example, Sleeping Beauty transposase recognition sites, or PiggyBac). The transposon gene cassette may be incorporated into an integrated genomic sequence or may exist freely as circular DNA. In some embodiments, the transposon gene cassette encodes a promoter, a CAR, and a signal sequence to direct the protein to the cell surface.

As used herein, “integrase vector systems” work by integrating a viral donor nucleic acid with specific attachment sites to a target genome. Through the use of integrase, the viral DNA is inserted into the host DNA.

As used herein, “T-cells” or “T lymphocytes” can be from any mammal, preferably a primate, including monkeys or humans, a companion animal such as a dog, cat, or horse, or a domestic animal, such as a sheep, goat, or cattle. In some alternatives the T-cells are allogeneic (from the same species but different donor) as the recipient subject; in some alternatives the T-cells are autologous (the donor and the recipient are the same); in some alternatives the T-cells arc syngeneic (the donor and the recipients are different but are identical twins).

As used herein, “T cell precursors” refers to lymphoid precursor cells that can migrate to the thymus and become T cell precursors, which do not express a T cell receptor. All T cells originate from hematopoietic stem cells in the bone marrow. Hematopoietic progenitors (lymphoid progenitor cells) from hematopoietic stem cells populate the thymus and expand by cell division to generate a large population of immature thymocytes. The earliest thymocytes express neither CD4 nor CD8 and are therefore classed as double-negative (CD4-CD8-) cells. As they progress through their development, they become double-positive thymocytes (CD4+CD8+), and finally mature to single-positive (CD4+CD8- or CD4-CD8+) thymocytes that are then released from the thymus to peripheral tissues.

As used herein, “hematopoietic stem cells” or “HSC” are precursor cells that can give rise to myeloid cells such as, for example, macrophages, monocytes, macrophages, neutrophils, basophils, eosinophils, erythrocytes, megakaryocytes/platelets, dendritic cells and/or lymphoid lineages (such as, for example, T-cells, B-cells, or NK-cells). HSCs have a heterogeneous population in which three classes of stem cells exist, which are distinguished by their ratio of lymphoid to myeloid progeny in the blood (L/M).

As used herein, “CD4+ expressing T-cell,” or “CD4+ T-cell,” are used synonymously throughout, is also known as T helper cells, which play an important role in the immune system, and in the adaptive immune system. CD4+ T-cells also help the activity of other immune cells by releasing T-cell cytokines. These cells help, suppress or regulate immune responses. They are essential in B cell antibody class switching, in the activation and growth of cytotoxic T-cells, and in maximizing bactericidal activity of phagocytes, such as macrophages. CD4+ expressing T-cells have the ability to make some cytokines, however the amounts of cytokines made by CD4+ T-cells are not at a concentration that promotes, improves, contributes to, or induces engraftment fitness. As described herein, “CD4+ T-cells” are mature T helper-cells that play a role in the adaptive immune system.

As used herein, “CD8+ expressing T-cell” or “CD8+ T-cell,” are used synonymously throughout, is also known as a TC, cytotoxic T lymphocyte, CTL, T-killer cell, cytolytic T-cell or killer T-cell. As described herein, CD8+ T-cells are T-lymphocytes that can kill cancer cells, virally infected cells, or damaged cells. CD8+ T-cells express T-cell receptors (TCRs) that can recognize a specific antigen. CD8+ T-cells express CD8 on the surface. CD8+ expressing T-cells have the ability to make some cytokines, however the amounts of cytokines made by CD8+ T-cells are not at a concentration that promotes, improves, contributes to, or induces engraftment fitness. “CD8 T-cells” or “killer T-cells” are T-lymphocytes that can kill cancer cells, cells that are infected with viruses or cells that are damaged.

Mature T cells express the surface protein CD4 and are referred to as CD4+ T-cells. CD4+ T-cells are generally treated as having a pre-defined role as helper T-cells within the immune system. For example, when an antigen-presenting cell expresses an antigen on MIIC class II, a CD4+ cell will aid those cells through a combination of cell to cell interactions (e.g. CD40 and CD40L) and through cytokines. Nevertheless, there are rare exceptions; for example, sub-groups of regulatory T-cells, natural killer cells, and cytotoxic T-cells express CD4. All of the latter CD4+ expressing T-cell groups are not considered T helper cells.

As used herein, “central memory” T-cell (or “TCM”) refers to an antigen experienced CTL that expresses CD62L or CCR-7 and CD45RO on the surface thereof and does not express or has decreased expression of CD45RA as compared to naïve cells. In some embodiments, central memory cells are positive for expression of CD62L, CCR7, CD28, CD127, CD45RO, and/or CD95, and have decreased expression of CD54RA, as compared to naïve cells.

As used herein, “effector memory” T-cell (or “TEM”) refers to an antigen experienced T-cell that does not express or has decreased expression of CD62L on the surface thereof as compared to central memory cells, and does not express or has decreased expression of CD45RA as compared to naïve cell. In some embodiments, effector memory cells are negative for expression of CD62L and/or CCR7, as compared to naïve cells or central memory cells, and have variable expression of CD28 and/or CD45RA.

As used herein, “naïve” T-cells refers to a non-antigen experienced T lymphocyte that expresses CD62L and/or CD45RA, and/or does not express CD45RO− as compared to central or effector memory cells. In some embodiments, naïve CD8+ T lymphocytes are characterized by the expression of phenotypic markers of naïve T-cells including CD62L, CCR7, CD28, CD127, or CD45RA.

As used herein, “effector” “TE” T-cells refers to a antigen experienced cytotoxic T lymphocyte cells that do not express or have decreased expression of CD62L, CCR7, CD28, and are positive for granzyme B or perforin or both, as compared to central memory or naïve T-cells.

As used herein, “engraftment fitness” has its plain and ordinary meaning when read in light of the specification, and includes, for example, the ability of a cell to grow and proliferate after the cells have entered the body, e.g., blood stream, through adoptive transfer. Engraftment can usually occur within two to four weeks after the transfer. Engraftment can be monitored by checking blood counts for a specific cell on a frequent basis. In some alternatives of the method of treating, inhibiting, or ameliorating a disease in a subject is provided, the method can comprise administering a composition or product combination comprising the genetically modified T-cells, as described herein. In some embodiments, the method can further comprise monitoring the subject by checking the blood counts for the genetically modified T-cells that expresses a chimeric antigen receptor e.g., by identifying the presence or absence of a marker associated with the transferred T-cells.

T-cells with improved engraftment fitness may have specific markers on the cell surface that confer generation and long-term maintenance of T-cell immunity. There are several proteins that are known for T-cell activation and survival. CD28 is a protein expressed on T-cells that provide co-stimulatory signals required for T-cell activation and survival. CD27 is required for generation and long-term maintenance of T-cell immunity. It binds to ligand CD70 and plays a key role in regulating B-cell activation and immunoglobulin synthesis. L-selectin, also known as CD62L is a cell adhesion molecule found on lymphocytes. L-selectin functions as a “homing receptor” for lymphocytes or T-cells to enter secondary lymphoid tissues via high endothelial venues. Ligands present on endothelial cells will bind to lymphocytes expressing L-selectin, slowing lymphocyte trafficking through the blood, and can facilitate entry into a secondary lymphoid organ at that point. In some embodiments, of the method of making genetically modified T-cells, which have a chimeric antigen receptor, the T-cells comprise at least one receptor that promotes, induces, improves, or contributes to engraftment fitness. In some embodiments, the at least one receptor is CD45 RA, CD45 RO, CCR7, CD25, CD127, CD57, CD137, CD27, CD28 or CD62L or any combination thereof. In some embodiments, the at least one receptor is CD27, CD28 or CD62L or any combination thereof. In some alternatives provided herein, methods are described in which CD4+ and CD8+ expressing T-cells are genetically modified by transduction of genetic material in the form of a vector such that the genetically modified CD4+ and CD8+ expressing T-cells expresses a specific chimeric antigen receptor. In some alternatives of the genetically modified CD4+ and CD8+ T-cells, the genetically modified CD4+ and CD8+ T-cells are further modified to improve or enhance engraftment fitness.

As used herein, “CD33,” has its plain and ordinary meaning and may refer to, for example, a transmembrane receptor expressed on cells of myeloid lineage. The extracellular portion of this receptor contains two immunoglobulin domains (one IgV and one IgC2 domain), placing CD33 within the immunoglobulin superfamily. The intracellular portion of CD33 contains immunoreceptor tyrosine-based inhibitory motifs (ITIMs) that are implicated in inhibition of cellular activity. CD33 is also a tumor associated antigen that is overexpressed on the surface of a variety of tumor cell types. CD33 has also been implicated in leukemias and lymphoma such as large B-cell lymphoma (Corean et al. Case Rep Hematol. 2018; 2018: 5320590; expressly incorporated by reference in its entirety).

As used herein, “protein” has its plain and ordinary meaning when read in light of the specification, and includes, for example, a macromolecule comprising one or more polypeptide chains. A protein can therefore comprise of peptides, which are chains of amino acid monomers linked by peptide (amide) bonds, formed by any one or more of the amino acids. A protein or peptide can contain at least two amino acids, and no limitation is placed on the maximum number of amino acids that can comprise the protein or peptide sequence. Without being limiting, the amino acids are, for example, arginine, histidine, lysine, aspartic acid, glutamic acid, serine, threonine, asparagine, glutamine, cysteine, cystine, glycine, proline, alanine, valine, hydroxyproline, isoleucine, leucine, pyrolysine, methionine, phenylalanine, tyrosine, tryptophan, ornithine, S-adenosylmethionine, or selenocysteine. A protein can also comprise non-peptide components, such as carbohydrate groups, for example. Carbohydrates and other non-peptide substituents can be added to a protein by the cell in which the protein is produced and will vary with the type of cell. Proteins are defined herein in terms of their amino acid backbone structures; substituents such as carbohydrate groups are generally not specified, but can be present nonetheless. In some embodiments, a CAR T-cell is also engineered to further express a protein, such as a cytokine, a chimeric cytokine receptor, a chimeric costimulatory molecule, a dominant negative receptor, an immunostimulatory molecule, or an immunoregulatory molecule.

As used herein, “cytokines” has its plain and ordinary meaning when read in light of the specification, and includes, for example, small proteins (5-25 kDa) that are important in cell signaling. Cytokines are released by cells and affect the behavior of other cells, and sometimes the releasing cell itself, such as a T-cell. Cytokines can include, for example, chemokines, interferons, interleukins, lymphokines, or tumor necrosis factor or any combination thereof. Cytokines can be produced by a broad range of cells, which can include, for example, immune cells like macrophages, B lymphocytes, T lymphocytes and mast cells, as well as, endothelial cells, fibroblasts, and various stromal cells.

Cytokines can act through receptors and are important in the immune system as the cytokines can modulate the balance between humoral and cell-based immune responses, and they can regulate the maturation, growth, and responsiveness of particular cell populations. Some cytokines enhance or inhibit the action of other cytokines in complex ways. Without being limiting, cytokines can include, for example, Acylation stimulating protein, Adipokine, Albinterferon, CCL1, CCL11, CCL12, CCL13, CCL14, CCL15, CCL16, CCL17, CCL18, CCL19, CCL2, CCL20, CCL21, CCL22, CCL23, CCL24, CCL25, CCL26, CCL27, CCL28, CCL3, CCL5, CCL6, CCL7, CCL8, CCL9, Chemokine, Colony-stimulating factor, CX3CL1, CX3CR1, CXCL1, CXCL10, CXCL11, CXCL13, CXCL14, CXCL15, CXCL16, CXCL17, CXCL2, CXCL3, CXCL5, CXCL6, CXCL7, CXCL9, Erythropoietin, Gc-MAF, Granulocyte colony-stimulating factor, Granulocyte macrophage colony-stimulating factor, Hepatocyte growth factor, IL 10 family of cytokines, IL 17 family of cytokines, IL1A, IL1B, Inflammasome, Interferome, Interferon, Interferon beta 1a, Interferon beta 1b, Interferon gamma, Interferon type I, Interferon type II, Interferon type III, Interleukin, Interleukin 1 family, Interleukin 1 receptor antagonist, Interleukin 10, Interleukin 12, Interleukin 12 subunit beta, Interleukin 13, Interleukin 15, Interleukin 16, Interleukin 2, Interleukin 23, Interleukin 23 subunit alpha, Interleukin 34, Interleukin 35, Interleukin 6, Interleukin 7, Interleukin 8, Interleukin 36, Leukemia inhibitory factor, Leukocyte-promoting factor, Lymphokine, Lymphotoxin, Lymphotoxin alpha, Lymphotoxin beta, Macrophage colony-stimulating factor, Macrophage inflammatory protein, Macrophage-activating factor, Monokine, Myokine, Myonectin, Nicotinamide phosphoribosyltransferase, Oncostatin M, Oprelvekin, Platelet factor 4, Proinflammatory cytokine, Promegapoietin, RANKL, Stromal cell-derived factor 1, Talimogene laherparepvec, Tumor necrosis factor alpha, Tumor necrosis factors, XCL1, XCL2, GM-CSF, or XCR1 or any combination thereof. In some alternatives of the methods of making genetically modified T-cells, a transduced population of CD8+ expressing T-cells and/or CD4+ expressing T-cells is contacted with at least one cytokine so as to generate a transduced, cytokine-stimulated population of CD8+ T-cells and/or CD4+ T-cells. In some alternatives, the at least one cytokine utilized comprises GM-CSF, IL-7, IL-12, IL-15, IL-18, IL-2 or IL-21 or any combination thereof. In some embodiments, the period of contact with the cytokine is at least one day, such as for at least 1, 2, 3, 4, 5, 6, 7, 8, 9, 10, 11, 12, 13, 14, 15, 16, 17, 18, 19, or 20 days or any time that is within a range of times defined by any two of the aforementioned time points.

As used herein, “interleukins” or IL are cytokines that the immune system depends largely upon. Examples of interleukins, which can be utilized herein, for example, include IL-1, IL-2, IL-3, IL-4, IL-5, IL-6, I1-7, IL-8/CXCL8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, or IL-36 or any combination thereof. Contacting T-cells with interleukins can have effects that promote, support, induce, or improve engraftment fitness of the cells. IL-1, for example can function in the maturation & proliferation of T-cells. IL-2, for example, can stimulate growth and differentiation of T-cell response. IL-3, for example, can promote differentiation and proliferation of myeloid progenitor cells. IL-4, for example, can promote proliferation and differentiation. IL-7, for example, can promote differentiation and proliferation of lymphoid progenitor cells, involved in B, T, and NK cell survival, development, and homeostasis. IL-15, for example, can induce production of natural killer cells. IL-21, for example, co-stimulates activation and proliferation of CD8+ T-cells, augments NK cytotoxicity, augments CD40-driven B cell proliferation, differentiation and isotype switching, and promotes differentiation of Thl7 cells.

As used herein, “propagating cells” or propagation refers to steps to allow proliferation, expansion, growth and reproduction of cells. For example, cultures of CD8+ T-cells and CD4+ T-cells can typically be incubated under conditions that are suitable for the growth and proliferation of T lymphocytes. In some alternatives of the method of making genetically modified T-cells, which have a chimeric antigen receptor, the CD4+ expressing T-cells are propagated for at least 1 day and may be propagated for 20 days, such as 1, 2, 3, 4, 5, 6, 7, 8, 9, 10, 11, 12, 13, 14, 15, 16, 17, 18, 19, or 20 days or for a period that is within a range defined by any two of the aforementioned time periods. In some alternatives of the method of making genetically modified T-cells, which have a chimeric antigen receptor, the CD8+ expressing T-cells are propagated for at least 1 day and may be propagated for 20 days, such as 1, 2, 3, 4, 5, 6, 7, 8, 9, 10, 11, 12, 13, 14, 15, 16, 17, 18, 19, or 20 days or for a period that is within a range defined by any two of the aforementioned time periods.

As used herein, “affinity selection,” refers to the selection of a specific molecule or cell having a selectable cell surface marker by binding to the molecule or marker or an epitope present thereon with a binding affinity agent, which allows for one to select out the specific molecule or cell of interest. Affinity selection can be performed by, for example, antibodies, conjugated antibodies, lectins, aptamers, or peptides or any combination thereof. In some embodiments, of the method of making genetically modified T-cells, the separating of the CD8+ population of T-cells and/or a CD4+ population of T-cells from a mixed population of T-cells is performed by affinity selection for T-cells having an epitope present on CD8 and/or CD4. In some alternatives of the method, anti-CD8 or anti-CD4 antibodies or binding portions thereof are used to select out the cells of interest. In some alternatives of the method, the separating of the CD8+ population of T-cells and/or a CD4+ population of T-cells from a mixed population of T-cells is performed by flow cytometry. In some alternatives of the method, the separating of the CD8+ population of T-cells and/or a CD4+ population of T-cells from a mixed population of T-cells is performed by immuno-magnetic selection. In some alternatives of the methods, the anti-CD8 or the anti-CD4 antibodies or binding fragments thereof are conjugated to a solid support such as, for example, an inert bead or an inert particle.

In another alternative, the expansion method or propagation can further comprise adding anti-CD3 and/or anti CD28 antibody to the culture medium (e.g., at a concentration of at least 0.5 ng/ml). In another alternative, the method of making genetically modified T-cells, which have a chimeric antigen receptor method can further comprise adding IL-2, IL-15, or IL-21 or any combination thereof to the culture medium (e.g., wherein the concentration of IL-2 is at least 10 units/ml). In another alternative, the method of making genetically modified T-cells, which have a chimeric antigen receptor method can further comprise adding IL-7, IL-15, or IL21 or any combination thereof to the culture medium (e.g., wherein the concentration of IL-2 is at least 10 units/ml). After isolation of T lymphocytes, both cytotoxic and helper T lymphocytes can be sorted into naïve, memory, and effector T-cell subpopulations either before or after expansion.

As used herein, “zinc finger nuclease” has its plain and ordinary meaning when read in light of the specification, and includes, for example, artificial restriction enzymes generated by fusing a zinc finger DNA-binding domain to a DNA-cleavage domain. Zinc finger domains can be engineered to target specific desired DNA sequences and this enables zinc-finger nucleases to target unique sequences within complex genomes. Methods for using zinc finger nucleases have been previously described (Bach et al. Biotechnol Res Int. 2014; 2014: 970595; expressly incorporated by reference in its entirety herein).

As used herein, “TALEN” or “Transcription activator-like effector nuclease” has its plain and ordinary meaning when read in light of the specification, and includes, for example, restriction enzymes that can be engineered to cut specific sequences of DNA. They are made by fusing a TAL effector DNA-binding domain to a DNA cleavage domain (a nuclease which cuts DNA strands). Transcription activator-like effectors (TALEs) can be engineered to bind practically any desired DNA sequence, so when combined with a nuclease, DNA can be cut at specific locations. The restriction enzymes can be introduced into cells, for use in gene editing or for genome editing in situ, a technique known as genome editing with engineered nucleases. Alongside zinc finger nucleases and CRISPR/Cas9, TALEN is a prominent tool in the field of genome editing. The correspondence between amino acids in TAL effectors and DNA bases in their target sites makes them useful for protein engineering applications. Artificial TAL effectors have been designed that are capable of recognizing new DNA sequences in a variety of experimental systems. Engineered TAL effectors may be used to create artificial transcription factors that can be used to target and activate or repress endogenous genes in human cells, for example. In some embodiments, MegaTalens, or MegaTals, are modular fusion proteins that combine homing endonucleases (meganucleases) and the DNA binding domains of TALENs (TAL effectors). Such monomeric protein fusion can increase the specificity of gene editing approaches and limits off-target DNA cleavage.

As used herein, “CRISPRs” (clustered regularly interspaced short palindromic repeats), has its plain and ordinary meaning when read in light of the specification, and includes, for example, segments of prokaryotic DNA containing short repetitions of base sequences. Each repetition is followed by short segments of “spacer DNA” from previous exposures to a bacterial virus or plasmid. The CRISPR/Cas system is a prokaryotic immune system that confers resistance to foreign genetic elements such as plasmids and phages and provides a form of acquired immunity. CRISPR spacers recognize and cut these exogenous genetic elements in a manner analogous to RNAi in eukaryotic organisms. CRISPR/Cas system has been used for gene editing (adding, disrupting or changing the sequence of specific genes) and gene regulation in species throughout the tree of life. By delivering the Cas9 protein and appropriate guide RNAs into a cell, the organism's genome can be cut at any desired location. One can use CRISPR to build RNA-guided gene editing tools capable of altering the genomes of entire populations.

As used herein, “genetically modified immune cells” or “Genetically engineered cells” are made by a process called genetic engineering, which can include but is not limited to manipulating a cell's own genome or inserting a new nucleic acid into a cell. In some embodiments, these cells can be macrophages and can also be referred to as genetically engineered macrophages (GEMs). These techniques can be used to change the genetic makeup of the cell and can include inserting a vector encoding a gene of interest into a cell, and genome editing using RNAi systems, meganucleases, zinc finger nucleases, transcription activator like effector nucleases (TALENS), or CRISPRs. Without being limiting, the vectors encoding the gene of interest can be a viral vector, DNA or an mRNA. In some embodiments, described herein, genetically modified immune cells are provided. In some embodiments, the genetically modified immune cells are made using genome editing proteins or systems, such as for example, meganucleases, zinc finger nucleases, transcription activator like effector nucleases (TALENS), CRISPR/VP64-Cas9 systems or CRISPR/CAS9 systems.

Some embodiments include polypeptide sequences or conservative variations thereof, such as conservative substitutions in a polypeptide sequence. In some embodiments, “conservative amino acid substitution” refers to amino acid substitutions that substitute functionally-equivalent amino acids. Conservative amino acid changes result in silent changes in the amino acid sequence of the resulting peptide. For example, one or more amino acids of a similar polarity act as functional equivalents and result in a silent alteration within the amino acid sequence of the peptide. Substitutions that are charge neutral and which replace a residue with a smaller residue may also be considered “conservative substitutions” even if the residues are in different groups (e.g., replacement of phenylalanine with the smaller isoleucine). Families of amino acid residues having similar side chains have been defined in the art. Several families of conservative amino acid substitutions are shown in TABLE 1.

TABLE 1

| Family | Amino Acids |
|---|---|
| non-polar | Trp, Phe, Met, Leu, Ile, Val, Ala, Pro |
| uncharged polar | Gly, Ser, Thr, Asn, Gln, Tyr, Cys |
| acidic/negatively charged | Asp, Glu |
| basic/positively charged | Arg, Lys, His |
| Beta-branched | Thr, Val, Ile |
| residues that influence chain | Gly, Pro |
| orientation | |
| aromatic | Trp, Tyr, Phe, His |

### Certain Polynucleotides

Some embodiments of the methods and compositions provided herein include a polynucleotide encoding a CAR, which is capable of specifically binding to CD33M (CD33 Iso-L protein or long isoform). In some such embodiments, the CAR is capable of specifically binding to a polypeptide and/or epitope encoded by exon 2 of the CD33 gene. In some embodiments, the polynucleotide comprises a first nucleic acid encoding an antibody or binding fragment thereof or a scFv, wherein the antibody or binding fragment thereof or scFv is capable of specifically binding to CD33M, a second nucleic acid encoding an extracellular spacer, such as a de-immunized extracellular spacer, a third nucleic acid encoding a transmembrane domain, a fourth nucleic acid encoding a signaling domain, wherein the signaling domain comprises a 4-1BB domain and/or CD3-zeta domain and a fifth nucleic acid encoding a linker. TABLE 2 lists certain sequences useful with embodiments provided herein.

In some embodiments, the polynucleotide also includes a sixth nucleic acid encoding a marker domain. In some embodiments, the marker sequence may encode a protein such as Her2tG or EGFRt. Marker sequences may be used for selection experiments or for enriching the cells that carry the CD33M (CD33 Iso-L protein or long isoform) specific CAR.

In some embodiments, the polynucleotide further comprises a seventh nucleic acid encoding a signal sequence for cell surface expression.

In some embodiments, the polynucleotide further comprises an inducible promoter sequence. In some embodiments, the promoter is inducible by a drug, such as tamoxifen.

In some embodiments, the spacer is selected for increased T cell proliferation and/or cytokine production in response to the ligand as compared to a reference chimeric receptor. In some embodiments, the spacer comprises a IgG4 hinge region or portion thereof (e.g., at least, equal to 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, or 55 amino acids or a length within a range defined by any two of the aforementioned lengths).

In some embodiments, the polynucleotide further comprises a seventh nucleic acid encoding a suicide gene system. It is well known that CAR T cell therapy may lead to cytokine release syndrome (CRS), which is also known as cytokine storm, neurotoxicities and cerebral edema, for example. As such it is of interest to develop CAR T cells with “off switches” so as to avoid potentially fatal side effects. In some embodiments, the suicide gene system is a Herpes Simplex Virus Thymidine Kinase (HSVTK)/Ganciclovir (GCV) suicide gene system or an inducible Caspase suicide gene system.

In some embodiments, the linker is a ribosome skip sequence or IRES sequence. In some embodiments, the ribosome skip sequence is a P2A, T2A, E2A or F2A sequence.

Some embodiments include a vector comprising a polynucleotide provided herein. In some embodiments, the vector is a viral vector. In some embodiments, the vector is a lentiviral vector, foamy viral vector, retroviral vector or a gammaretroviral vector. In some embodiments, the vector is a transposon, integrase vector system, or an mRNA vector.

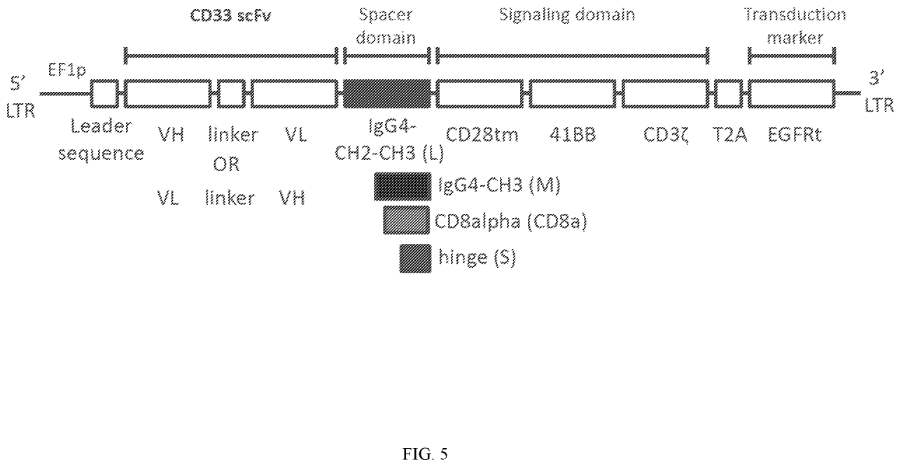

Some embodiments of the methods and compositions provided herein include a polynucleotide encoding a CD33 targeting CAR. Some embodiments include a polynucleotide represented in FIG. 5. In some embodiments, the polynucleotide encodes a scFv comprising a VH-CDR1, VH-CDR2, and/or a VH-CDR3 having at least 95, 96, 97, 98, or 99% sequence identity to the VH-CDR1, VH-CDR2, and/or a VH-CDR3 amino acid sequences listed in TABLE 2. In some embodiments, the scFv comprises a VH-CDR1, VH-CDR2, or a VH-CDR3 or any combination thereof having the amino acid sequences listed in TABLE 2. In some embodiments, the polynucleotide encodes a scFv comprising a VL-CDR1, VL-CDR2, or a VL-CDR3 having at least 95, 96, 97, 98, or 99% sequence identity to the any one or more of the amino acid sequences listed in TABLE 2. In some embodiments, the scFv comprises a VL-CDR1, VL-CDR2, or a VL-CDR3 or any combination thereof having the amino acid sequences listed in TABLE 2.

In some embodiments, the polynucleotide encodes a scFv comprising a VH domain, having at least 95, 96, 97, 98, or 99% sequence identity to any one or more of the VH domain amino acid sequences listed in TABLE 2. In some embodiments, the scFv comprises a VH domain having the VH domain amino acid sequence listed in TABLE 2. In some embodiments, the polynucleotide encodes a scFv comprising a VL domain, having at least 95, 96, 97, 98, or 99% sequence identity to any one or more of the VL domain amino acid sequences listed in TABLE 2. In some embodiments, the scFv comprises a VL domain having the VL domain amino acid sequence listed in TABLE 2.

In some embodiments, the polynucleotide encodes a scFv comprising a VH domain and a VL domain linked via a linker. In some embodiments the VH domain and VL domain are in a VH-VL orientation with respect to the N-terminus of a polypeptide. In some embodiments the VH domain and VL domain are in a VL-VH orientation with respect to the N-terminus of a polypeptide.

In some embodiments, the polypeptide encodes a spacer, such as a short spacer, a medium spacer, or a long spacer. In some embodiments, the spacer has at least 95, 96, 97, 98, or 99% sequence identity to any one or more of the amino acid sequences of a spacer listed in TABLE 2. In some embodiments, the spacer comprises the amino acid sequence of an example spacer listed in TABLE 2.

TABLE 2

| Feature | |
|---|---|
| SEQ ID NO | Sequence |
| M (medium) | GGCCAGCCTAGAGAACCCCAGGTGTACACCCTGCCTCCCAGC |
| spacer | CAGGAAGAGATGACCAAGAACCAGGTGTCCCTGACCTGCCTG |
| SEQ ID NO: 01 | GTCAAAGGCTTCTACCCCAGCGATATCGCCGTGGAATGGGAG |
| | AGCAACGGCCAGCCCGAGAACAACTACAAGACCACCCCCCCT |
| | GTGCTGGACAGCGACGGCAGCTTCTTCCTGTACTCCCGGCTGA |
| | CCGTGGACAAGAGCCGGTGGCAGGAAGGCAACGTCTTCAGCT |
| | GCAGCGTGATGCACGAGGCCCTGCACAACCACTACACCCAGA |
| | AGTCCCTGAGCCTGAGCCTGGGCAAG |
| L (long) spacer | GCCCCCGAGTTCGACGGCGGACCCAGCGTGTTCCTGTTCCCCC |
| SEQ ID NO: 02 | CCAAGCCCAAGGACACCCTGATGATCAGCCGGACCCCCGAGG |
| | TGACCTGCGTGGTGGTGGACGTGAGCCAGGAAGATCCCGAGG |
| | TCCAGTTCAATTGGTACGTGGACGGCGTGGAAGTGCACAACG |
| | CCAAGACCAAGCCCAGAGAGGAACAGTTCCAGAGCACCTACC |
| | GGGTGGTGTCTGTGCTGACCGTGCTGCACCAGGACTGGCTGAA |
| | CGGCAAAGAATACAAGTGCAAGGTGTCCAACAAGGGCCTGCC |
| | CAGCAGCATCGAAAAGACCATCAGCAAGGCCAAGGGCCAGCC |
| | TCGCGAGCCCCAGGTGTACACCCTGCCTCCCTCCCAGGAAGAG |
| | ATGACCAAGAACCAGGTGTCCCTGACCTGCCTGGTGAAGGGC |
| | TTCTACCCCAGCGACATCGCCGTGGAGTGGGAGAGCAACGGC |
| | CAGCCTGAGAACAACTACAAGACCACCCCTCCCGTGCTGGAC |
| | AGCGACGGCAGCTTCTTCCTGTACAGCCGGCTGACCGTGGACA |
| | AGAGCCGGTGGCAGGAAGGCAACGTCTTTAGCTGCAGCGTGA |
| | TGCACGAGGCCCTGCACAACCACTACACCCAGAAGAGCCTGA |
| | GCCTGTCCCTGGGCAAG |
| M (medium) | MTKNQVSLTCLVKGFYPSDIAVEWESNGQPENNYKTTPPVLDSD |
| spacer | GSFFLYSRLTVDKSRWQEGNVFSCSVMHEALHNHYTQKSLSLSL |
| SEQ ID NO: 03 | GK |
| L (long) spacer | MISRTPEVTCVVVDVSQEDPEVQFNWYVDGVEVHNAKTKPREE |
| SEQ ID NO: 04 | QFQSTYRVVSVLTVLHQDWLNGKEYKCKVSNKGLPSSIEKTISK |
| | AKGQPREPQVYTLPPSQEEMTKNQVSLTCLVKGFYPSDIAVEWE |
| | SNGQPENNYKTTPPVLDSDGSFFLYSRLTVDKSRWQEGNVFSCS |
| | VMHEALHNHYTQKSLSLSLGK |
| Anti-CD33 VH | QVQLVQSGAEVKKPGASVKVSCKASGYTFTNYDINWVRQAPGQ |
| domain | GLEWIGWIYPGDGSTKYNEKFKAKATLTADTSTSTAYMELRSLR |
| SEQ ID NO: 05 | SDDTAVYYCASGYEDAMDYWGQGTTVTVSS |
| VH-CDR1 | NYDIN |
| SEQ ID NO: 06 | |
| VH-CDR2 | WIYPGDGSTKYNEKFKA |
| SEQ ID NO: 07 | |
| VH-CDR3 | GYEDAMDY |
| SEQ ID NO: 08 | |
| Anti-CD33 VL | DIQMTQSPSSLSASVGDRVTINCKASQDINSYLSWFQQKPGKAPK |
| domain | TLIYRANRLVDGVPSRFSGSGSGQDYTLTISSLQPEDFATYYCLQ |
| SEQ ID NO: 09 | YDEFPLTFGGGTKVEIK |
| VL-CDR1 | KASQDINSYLS |
| SEQ ID NO: 10 | |
| VL-CDR2 | RANNRLVD |
| SEQ ID NO: 11 | |
| VL-CDR3 | LQYDEFPLT |
| SEQ ID NO: 12 | |
| scFv linker | GGGGSGGGGSGGGGS |
| SEQ ID NO: 13 | |
| S (small) spacer | ESKYGPPCPPCP |
| SEQ ID NO: 14 | |
| S (small) spacer | GAATCTAAGTACGGACCGCCCTGCCCCCCTTGCCCT |
| SEQ ID NO: 15 | |
| M (medium) | ESKYGPPCPPCPGQPREPQVYTLPPSQEEMTKNQVSLTCLVKGFY |
| spacer | PSDIAVEWESNGQPENNYKTTPPVLDSDGSFFLYSRLTVDKSRW |
| SEQ ID NO: 16 | QEGNVFSCSVMHEALHNHYTQKSLSLSLGK |
| M (medium) | GAATCTAAGTACGGACCGCCCTGCCCCCCTTGCCCTGGCCAGC |
| spacer | CTAGAGAACCCCAGGTGTACACCCTGCCTCCCAGCCAGGAAG |
| SEQ ID NO: 17 | AGATGACCAAGAACCAGGTGTCCCTGACCTGCCTGGTCAAAG |
| | GCTTCTACCCCAGCGATATCGCCGTGGAATGGGAGAGCAACG |
| | GCCAGCCCGAGAACAACTACAAGACCACCCCCCCTGTGCTGG |
| | ACAGCGACGGCAGCTTCTTCCTGTACTCCCGGCTGACCGTGGA |
| | CAAGAGCCGGTGGCAGGAAGGCAACGTCTTCAGCTGCAGCGT |
| | GATGCACGAGGCCCTGCACAACCACTACACCCAGAAGTCCCT |
| | GAGCCTGAGCCTGGGCAAG |
| L (long) spacer | ESKYGPPCPPCPAPEFLGGPSVFLFPPKPKDTLMISRTPEVTCVVV |
| SEQ ID NO: 18 | DVSQEDPEVQFNWYVDGVEVHNAKTKPREEQFNSTYRVVSVLT |
| | VLHQDWLNGKEYKCKVSNKGLPSSIEKTISKAKGQPREPQVYTL |
| | PPSQEEMTKNQVSLTCLVKGFYPSDIAVEWESNGQPENNYKTTPP |
| | VLDSDGSFFLYSRLTVDKSRWQEGNVFSCSVMHEALHNHYTQK |
| | SLSLSLGK |
| L (long) spacer | ATCTAAGTACGGACCGCCCTGCCCCCCTTGCCCTGCCCCCGAG |
| SEQ ID NO: 19 | TTCGACGGCGGACCCAGCGTGTTCCTGTTCCCCCCCAAGCCCA |
| | AGGACACCCTGATGATCAGCCGGACCCCCGAGGTGACCTGCG |
| | TGGTGGTGGACGTGAGCCAGGAAGATCCCGAGGTCCAGTTCA |
| | ATTGGTACGTGGACGGCGTGGAAGTGCACAACGCCAAGACCA |
| | AGCCCAGAGAGGAACAGTTCCAGAGCACCTACCGGGTGGTGT |
| | CTGTGCTGACCGTGCTGCACCAGGACTGGCTGAACGGCAAAG |
| | AATACAAGTGCAAGGTGTCCAACAAGGGCCTGCCCAGCAGCA |
| | TCGAAAAGACCATCAGCAAGGCCAAGGGCCAGCCTCGCGAGC |
| | CCCAGGTGTACACCCTGCCTCCCTCCCAGGAAGAGATGACCA |
| | AGAACCAGGTGTCCCTGACCTGCCTGGTGAAGGGCTTCTACCC |
| | CAGCGACATCGCCGTGGAGTGGGAGAGCAACGGCCAGCCTGA |
| | GAACAACTACAAGACCACCCCTCCCGTGCTGGACAGCGACGG |
| | CAGCTTCTTCCTGTACAGCCGGCTGACCGTGGACAAGAGCCGG |
| | TGGCAGGAAGGCAACGTCTTTAGCTGCAGCGTGATGCACGAG |
| | GCCCTGCACAACCACTACACCCAGAAGAGCCTGAGCCTGTCC |
| | CTGGGCAAG |
| CD28tm | MFWVLVVVGGVLACYSLLVTVAFIIFWV |
| SEQ ID NO: 20 | |
| CD28tm | ATGTTCTGGGTGCTGGTGGTGGTCGGAGGCGTGCTGGCCTGCT |
| SEQ ID NO: 21 | ACAGCCTGCTGGTCACCGTGGCCTTCATCATCTTTTGGGTG |
| 41-BB | AAACGGGGCAGAAAGAAACTCCTGTATATATTCAAACAACCA |
| SEQ ID NO: 22 | TTTATGAGACCAGTACAAACTACTCAAGAGGAAGATGGCTGT |
| | AGCTGCCGATTTCCAGAAGAAGAAGAAGGAGGATGTGAACTG |
| CD3ζ | CGGGTGAAGTTCAGCAGAAGCGCCGACGCCCCTGCCTACCAG |
| SEQ ID NO: 23 | CAGGGCCAGAATCAGCTGTACAACGAGCTGAACCTGGGCAGA |
| | AGGGAAGAGTACGACGTCCTGGATAAGCGGAGAGGCCGGGA |
| | CCCTGAGATGGGCGGCAAGCCTCGGCGGAAGAACCCCCAGGA |
| | AGGCCTGTATAACGAACTGCAGAAAGACAAGATGGCCGAGGC |
| | CTACAGCGAGATCGGCATGAAGGGCGAGCGGAGGCGGGGCA |
| | AGGGCCACGACGGCCTGTATCAGGGCCTGTCCACCGCCACCA |
| | AGGATACCTACGACGCCCTGCACATGCAGGCCCTGCCCCCAA |
| | GG |
| T2A | CTCGAGGGCGGCGGAGAGGGCAGAGGAAGTCTTCTAACATGC |
| SEQ ID NO: 24 | GGTGACGTGGAGGAGAATCCCGGCCCTAGG |
| EGFRt | CGCAAAGTGTGTAACGGAATAGGTATTGGTGAATTTAAAGAC |
| SEQ ID NO: 25 | TCACTCTCCATAAATGCTACGAATATTAAACACTTCAAAAACT |
| | GCACCTCCATCAGTGGCGATCTCCACATCCTGCCGGTGGCATT |
| | TAGGGGTGACTCCTTCACACATACTCCTCCTCTGGATCCACAG |
| | GAACTGGATATTCTGAAAACCGTAAAGGAAATCACAGGGTTT |
| | TTGCTGATTCAGGCTTGGCCTGAAAACAGGACGGACCTCCATG |
| | CCTTTGAGAACCTAGAAATCATACGCGGCAGGACCAAGCAAC |
| | ATGGTCAGTTTTCTCTTGCAGTCGTCAGCCTGAACATAACATC |
| | CTTGGGATTACGCTCCCTCAAGGAGATAAGTGATGGAGATGT |
| | GATAATTTCAGGAAACAAAAATTTGTGCTATGCAAATACAAT |
| | AAACTGGAAAAAACTGTTTGGGACCTCCGGTCAGAAAACCAA |
| | AATTATAAGCAACAGAGGTGAAAACAGCTGCAAGGCCACAGG |
| | CCAGGTCTGCCATGCCTTGTGCTCCCCCGAGGGCTGCTGGGGC |
| | CCGGAGCCCAGGGACTGCGTCTCTTGCCGGAATGTCAGCCGA |
| | GGCAGGGAATGCGTGGACAAGTGCAACCTTCTGGAGGGTGAG |
| | CCAAGGGAGTTTGTGGAGAACTCTGAGTGCATACAGTGCCAC |
| | CCAGAGTGCCTGCCTCAGGCCATGAACATCACCTGCACAGGA |
| | CGGGGACCAGACAACTGTATCCAGTGTGCCCACTACATTGAC |
| | GGCCCCCACTGCGTCAAGACCTGCCCGGCAGGAGTCATGGGA |
| | GAAAACAACACCCTGGTCTGGAAGTACGCAGACGCCGGCCAT |
| | GTGTGCCACCTGTGCCATCCAAACTGCACCTACGGATGCACTG |
| | GGCCAGGTCTTGAAGGCTGTCCAACGAATGGGCCTAAGATCC |
| | CGTCCATCGCCACTGGGATGGTGGGGGCCCTCCTCTTGCTGCT |
| | GGTGGTGGCCCTGGGGATCGGCCTCTTCATG |
| DHFRdm | MVGSLNCIVAVSQNMGIGKNGDFPWPPLRNESRYFQRMTTTSSV |
| SEQ ID NO: 26 | EGKQNLVIMGKKTWFSIPEKNRPLKGRINLVLSRELKEPPQGAHF |
| | LSRSLDDALKLTEQPELANKVDMVWIVGGSSVYKEAMNHPGHL |
| | KLFVTRIMQDFESDTFFPEIDLEKYKLLPEYPGVLSDVQEEKGIKY |
| | KFEVYEKND |
| DHFRdm | ATGGTTGGTTCGCTAAACTGCATCGTCGCTGTGTCCCAGAACA |
| SEQ ID NO: 27 | TGGGCATCGGCAAGAACGGGGACTTCCCCTGGCCACCGCTCA |
| | GGAATGAATCCAGATATTTCCAGAGAATGACCACAACCTCTTC |
| | AGTAGAAGGTAAACAGAATCTGGTGATTATGGGTAAGAAGAC |
| | CTGGTTCTCCATTCCTGAGAAGAATCGACCTTTAAAGGGTAGA |
| | ATTAATTTAGTTCTCAGCAGAGAACTCAAGGAACCTCCACAAG |
| | GAGCTCATTTTCTTTCCAGAAGTCTAGATGATGCCTTAAAACT |
| | TACTGAACAACCAGAATTAGCAAATAAAGTAGACATGGTCTG |
| | GATAGTTGGTGGCAGTTCTGTTTATAAGGAAGCCATGAATCAC |
| | CCAGGCCATCTTAAACTATTTGTGACAAGGATCATGCAAGACT |
| | TTGAAAGTGACACGTTTTTTCCAGAAATTGATTTGGAGAAATA |
| | TAAACTTCTGCCAGAATACCCAGGTGTTCTCTCTGATGTCCAG |
| | GAGGAGAAAGGCATTAAGTACAAATTTGAAGTATATGAGAAG |
| | AATGATTAA |
| CD19 signal | MPPPRLLFFLLFLTP |
| sequence | |
| SEQ ID NO: 28 | |
| GMCSFR | MLLLVTSLLLCELPHPAFLLIP |
| signal sequence | |
| SEQ ID NO: 29 | |
| CD19t | ATGCCACCTCCTCGCCTCCTCTTCTTCCTCCTCTTCCTCACCCC |
| SEQ ID NO: 30 | CATGGAAGTCAGGCCCGAGGAACCTCTAGTGGTGAAGGTGGA |
| | AGAGGGAGATAACGCTGTGCTGCAGTGCCTCAAGGGGACCTC |
| | AGATGGCCCCACTCAGCAGCTGACCTGGTCTCGGGAGTCCCCG |
| | CTTAAACCCTTCTTAAAACTCAGCCTGGGGCTGCCAGGCCTGG |
| | GAATCCACATGAGGCCCCTGGCCATCTGGCTTTTCATCTTCAA |
| | CGTCTCTCAACAGATGGGGGGCTTCTACCTGTGCCAGCCGGGG |
| | CCCCCCTCTGAGAAGGCCTGGCAGCCTGGCTGGACAGTCAATG |
| | TGGAGGGCAGCGGGGAGCTGTTCCGGTGGAATGTTTCGGACC |
| | TAGGTGGCCTGGGCTGTGGCCTGAAGAACAGGTCCTCAGAGG |
| | GCCCCAGCTCCCCTTCCGGGAAGCTCATGAGCCCCAAGCTGTA |
| | TGTGTGGGCCAAAGACCGCCCTGAGATCTGGGAGGGAGAGCC |
| | TCCGTGTGTCCCACCGAGGGACAGCCTGAACCAGAGCCTCAG |
| | CCAGGACCTCACCATGGCCCCTGGCTCCACACTCTGGCTGTCC |
| | TGTGGGGTACCCCCTGACTCTGTGTCCAGGGGCCCCCTCTCCT |
| | GGACCCATGTGCACCCCAAGGGGCCTAAGTCATTGCTGAGCCT |
| | AGAGCTGAAGGACGATCGCCCGGCCAGAGATATGTGGGTAAT |
| | GGAGACGGGTCTGTTGTTGCCCCGGGCCACAGCTCAAGACGCT |
| | GGAAAGTATTATTGTCACCGTGGCAACCTGACCATGTCATTCC |
| | ACCTGGAGATCACTGCTCGGCCAGTACTATGGCACTGGCTGCT |
| | GAGGACTGGTGGCTGGAAGGTCTCAGCTGTGACTTTGGCTTAT |
| | CTGATCTTCTGCCTGTGTTCCCTTGTGGGCATTCTTCATCTTCA |
| | AAGAGCCCTGGTCCTGAGGAGGAAAAGATAA |
| CD19t | MPPPRLLFFLLFLTPMEVRPEEPLVVKVEEGDNAVLQCLKGTSDG |
| SEQ ID NO: 31 | PTQQLTWSRESPLKPFLKLSLGLPGLGIHMRPLAIWLFIFNVSQQM |
| | GGFYLCQPGPPSEKAWQPGWTVNVEGSGELFRWNVSDLGGLGC |
| | GLKNRSSEGPSSPSGKLMSPKLYVWAKDRPEIWEGEPPCVPPRDS |
| | LNQSLSQDLTMAPGSTLWLSCGVPPDSVSRGPLSWTHVHPKGPK |
| | SLLSLELKDDRPARDMWVMETGLLLPRATAQDAGKYYCHRGNL |
| | TMSFHLEITARPVLWHWLLRTGGWKVSAVTLAYLIFCLCSLVGIL |
| | HLQRALVLRRKR |
| GM-CSF | ATGCTTCTCCTGGTGACAAGCCTTCTGCTCTGTGAGTTACCAC |
| receptor-chain | ACCCAGCATTCCTCCTGATCCCA |
| signal sequence | |
| SEQ ID NO: 32 | |
| GM-CSF | MLLLVTSLLLCELPHPAFLLIP |
| receptor-chain | |
| signal sequence | |
| SEQ ID NO: 33 | |
| pJ03975_CD33 | ATGCTTCTCCTGGTGACAAGCCTTCTGCTCTGTGAGTTACCAC |
| VH-VL- | ACCCAGCATTCCTCCTGATCCCACAGGTTCAGCTGGTGCAGTC |
| IgG4Hinge- | TGGAGCTGAGGTGAAGAAGCCTGGGGCCTCAGTGAAGGTCTC |
| CD28tm-4- | CTGCAAGGCTTCTGGTTACACCTTTACCAATTATGATATAAAT |
| 1BB-T2A- | TGGGTGAGACAGGCCCCTGGACAAGGGCTTGAGTGGATTGGA |
| EGFRt_epHIV72 | TGGATTTATCCTGGAGATGGTAGTACCAAATATAATGAGAAAT |
| SEQ ID NO: 34 | TCAAGGCCAAGGCTACCCTGACAGCTGACACATCCACCAGCA |
| | CAGCCTACATGGAGCTGAGGAGCCTGAGATCTGATGACACAG |
| | CTGTGTATTACTGTGCTTCTGGATATGAAGATGCTATGGACTA |
| | CTGGGGGCAAGGGACCACAGTCACAGTCTCCTCAGGTGGCGG |
| | TGGCAGCGGCGGTGGTGGTTCCGGAGGCGGCGGTTCTGACAT |
| | CCAGATGACCCAGTCTCCATCCTCACTGTCTGCATCTGTAGGA |
| | GACAGAGTCACCATCAATTGTAAGGCTAGTCAGGACATTAAT |
| | AGCTATTTGAGCTGGTTTCAGCAGAAACCAGGGAAAGCCCCT |
| | AAGACCCTGATCTATAGAGCAAATAGATTGGTAGATGGGGTC |
| | CCATCAAGGTTCTCTGGCAGTGGATCTGGGCAAGATTATACTC |
| | TCACCATCAGCAGCCTGCAGCCTGAAGATTTTGCAACTTATTA |
| | CTGCTTGCAGTATGATGAGTTTCCTCTCACATTTGGAGGAGGG |
| | ACCAAGGTGGAGATCAAAGAATCTAAGTACGGACCGCCCTGC |
| | CCCCCTTGCCCTATGTTCTGGGTGCTGGTGGTGGTCGGAGGCG |
| | TGCTGGCCTGCTACAGCCTGCTGGTCACCGTGGCCTTCATCAT |
| | CTTTTGGGTGAAACGGGGCAGAAAGAAACTCCTGTATATATTC |
| | AAACAACCATTTATGAGACCAGTACAAACTACTCAAGAGGAA |
| | GATGGCTGTAGCTGCCGATTTCCAGAAGAAGAAGAAGGAGGA |
| | TGTGAACTGCTCGAGGGCGGCGGAGAGGGCAGAGGAAGTCTT |
| | CTAACATGCGGTGACGTGGAGGAGAATCCCGGCCCTAGGATG |
| | CTTCTCCTGGTGACAAGCCTTCTGCTCTGTGAGTTACCACACCC |
| | AGCATTCCTCCTGATCCCACGCAAAGTGTGTAACGGAATAGGT |
| | ATTGGTGAATTTAAAGACTCACTCTCCATAAATGCTACGAATA |
| | TTAAACACTTCAAAAACTGCACCTCCATCAGTGGCGATCTCCA |
| | CATCCTGCCGGTGGCATTTAGGGGTGACTCCTTCACACATACT |
| | CCTCCTCTGGATCCACAGGAACTGGATATTCTGAAAACCGTAA |
| | AGGAAATCACAGGGTTTTTGCTGATTCAGGCTTGGCCTGAAAA |
| | CAGGACGGACCTCCATGCCTTTGAGAACCTAGAAATCATACGC |
| | GGCAGGACCAAGCAACATGGTCAGTTTTCTCTTGCAGTCGTCA |
| | GCCTGAACATAACATCCTTGGGATTACGCTCCCTCAAGGAGAT |
| | AAGTGATGGAGATGTGATAATTTCAGGAAACAAAAATTTGTG |
| | CTATGCAAATACAATAAACTGGAAAAAACTGTTTGGGACCTCC |
| | GGTCAGAAAACCAAAATTATAAGCAACAGAGGTGAAAACAGC |
| | TGCAAGGCCACAGGCCAGGTCTGCCATGCCTTGTGCTCCCCCG |
| | AGGGCTGCTGGGGCCCGGAGCCCAGGGACTGCGTCTCTTGCC |
| | GGAATGTCAGCCGAGGCAGGGAATGCGTGGACAAGTGCAACC |
| | TTCTGGAGGGTGAGCCAAGGGAGTTTGTGGAGAACTCTGAGT |
| | GCATACAGTGCCACCCAGAGTGCCTGCCTCAGGCCATGAACAT |
| | CACCTGCACAGGACGGGGACCAGACAACTGTATCCAGTGTGC |
| | CCACTACATTGACGGCCCCCACTGCGTCAAGACCTGCCCGGCA |
| | GGAGTCATGGGAGAAAACAACACCCTGGTCTGGAAGTACGCA |
| | GACGCCGGCCATGTGTGCCACCTGTGCCATCCAAACTGCACCT |
| | ACGGATGCACTGGGCCAGGTCTTGAAGGCTGTCCAACGAATG |
| | GGCCTAAGATCCCGTCCATCGCCACTGGGATGGTGGGGGCCCT |
| | CCTCTTGCTGCTGGTGGTGGCCCTGGGGATCGGCCTCTTCATG |
| | TGA |
| pJ03975_CD33 | MLLLVTSLLLCELPHPAFLLIPQVQLVQSGAEVKKPGASVKVSCK |
| VH-VL- | ASGYTFTNYDINWVRQAPGQGLEWIGWIYPGDGSTKYNEKFKA |
| IgG4Hinge- | KATLTADTSTSTAYMELRSLRSDDTAVYYCASGYEDAMDYWGQ |
| CD28tm-4- | GTTVTVSSGGGGSGGGGSGGGGSDIQMTQSPSSLSASVGDRVTIN |
| 1BB-T2A- | CKASQDINSYLSWFQQKPGKAPKTLIYRANRLVDGVPSRFSGSGS |
| EGFRt_epHIV72 | GQDYTLTISSLQPEDFATYYCLQYDEFPLTFGGGTKVEIKESKYGP |
| SEQ ID NO: 35 | PCPPCPMFWVLVVVGGVLACYSLLVTVAFIIFWVKRGRKKLLYIF |
| | KQPFMRPVQTTQEEDGCSCRFPEEEEGGCELLEGGGEGRGSLLTC |
| | GDVEENPGPRMLLLVTSLLLCELPHPAFLLIPRKVCNGIGIGEFKD |
| | SLSINATNIKHFKNCTSISGDLHILPVAFRGDSFTHTPPLDPQELDIL |
| | KTVKEITGFLLIQAWPENRTDLHAFENLEIIRGRTKQHGQFSLAVV |
| | SLNITSLGLRSLKEISDGDVIISGNKNLCYANTINWKKLFGTSGQK |
| | TKIISNRGENSCKATGQVCHALCSPEGCWGPEPRDCVSCRNVSRG |
| | RECVDKCNLLEGEPREFVENSECIQCHPECLPQAMNITCTGRGPD |
| | NCIQCAHYIDGPHCVKTCPAGVMGENNTLVWKYADAGHVCHLC |
| | HPNCTYGCTGPGLEGCPTNGPKIPSIATGMVGALLLLLVVALGIG |
| | LFM |
| pJ04043- | ATGCTTCTCCTGGTGACAAGCCTTCTGCTCTGTGAGTTACCAC |
| CD33scFv-VH- | ACCCAGCATTCCTCCTGATCCCACAGGTTCAGCTGGTGCAGTC |
| VL-CD8hinge- | TGGAGCTGAGGTGAAGAAGCCTGGGGCCTCAGTGAAGGTCTC |
| tm-41BB-zeta- | CTGCAAGGCTTCTGGTTACACCTTTACCAATTATGATATAAAT |
| T2A-EGFRt- | TGGGTGAGACAGGCCCCTGGACAAGGGCTTGAGTGGATTGGA |
| epHIV72 | TGGATTTATCCTGGAGATGGTAGTACCAAATATAATGAGAAAT |
| SEQ ID NO: 36 | TCAAGGCCAAGGCTACCCTGACAGCTGACACATCCACCAGCA |
| | CAGCCTACATGGAGCTGAGGAGCCTGAGATCTGATGACACAG |
| | CTGTGTATTACTGTGCTTCTGGATATGAAGATGCTATGGACTA |
| | CTGGGGGCAAGGGACCACAGTCACAGTCTCCTCAGGTGGCGG |
| | TGGCAGCGGCGGTGGTGGTTCCGGAGGCGGCGGTTCTGACAT |
| | CCAGATGACCCAGTCTCCATCCTCACTGTCTGCATCTGTAGGA |
| | GACAGAGTCACCATCAATTGTAAGGCTAGTCAGGACATTAAT |
| | AGCTATTTGAGCTGGTTTCAGCAGAAACCAGGGAAAGCCCCT |
| | AAGACCCTGATCTATAGAGCAAATAGATTGGTAGATGGGGTC |
| | CCATCAAGGTTCTCTGGCAGTGGATCTGGGCAAGATTATACTC |
| | TCACCATCAGCAGCCTGCAGCCTGAAGATTTTGCAACTTATTA |
| | CTGCTTGCAGTATGATGAGTTTCCTCTCACATTTGGAGGAGGG |
| | ACCAAGGTGGAGATCAAAGAATCTAAGTACGGACCGGCCAAG |
| | CCTACCACCACCCCTGCCCCTAGACCTCCAACACCCGCCCCAA |
| | CAATCGCCAGCCAGCCTCTGTCTCTGAGGCCCGAGGCTTGTAG |
| | ACCAGCTGCTGGCGGAGCCGTGCACACCAGAGGACTGGATTT |
| | CGCCTGCGACATCTACATCTGGGCCCCTCTGGCCGGCACATGT |
| | GGCGTGCTGCTGCTGAGCCTCGTGATCACCAAACGGGGCAGA |
| | AAGAAACTCCTGTATATATTCAAACAACCATTTATGAGACCAG |
| | TACAAACTACTCAAGAGGAAGATGGCTGTAGCTGCCGATTTCC |
| | AGAAGAAGAAGAAGGAGGATGTGAACTGCGGGTGAAGTTCA |
| | GCAGAAGCGCCGACGCCCCTGCCTACCAGCAGGGCCAGAATC |
| | AGCTGTACAACGAGCTGAACCTGGGCAGAAGGGAAGAGTACG |
| | ACGTCCTGGATAAGCGGAGAGGCCGGGACCCTGAGATGGGCG |
| | GCAAGCCTCGGCGGAAGAACCCCCAGGAAGGCCTGTATAACG |
| | AACTGCAGAAAGACAAGATGGCCGAGGCCTACAGCGAGATCG |
| | GCATGAAGGGCGAGCGGAGGCGGGGCAAGGGCCACGACGGC |
| | CTGTATCAGGGCCTGTCCACCGCCACCAAGGATACCTACGACG |
| | CCCTGCACATGCAGGCCCTGCCCCCAAGGCTCGAGGGCGGCG |
| | GAGAGGGCAGAGGAAGTCTTCTAACATGCGGTGACGTGGAGG |
| | AGAATCCCGGCCCTAGGATGCTTCTCCTGGTGACAAGCCTTCT |
| | GCTCTGTGAGTTACCACACCCAGCATTCCTCCTGATCCCACGC |
| | AAAGTGTGTAACGGAATAGGTATTGGTGAATTTAAAGACTCA |
| | CTCTCCATAAATGCTACGAATATTAAACACTTCAAAAACTGCA |
| | CCTCCATCAGTGGCGATCTCCACATCCTGCCGGTGGCATTTAG |
| | GGGTGACTCCTTCACACATACTCCTCCTCTGGATCCACAGGAA |
| | CTGGATATTCTGAAAACCGTAAAGGAAATCACAGGGTTTTTGC |
| | TGATTCAGGCTTGGCCTGAAAACAGGACGGACCTCCATGCCTT |
| | TGAGAACCTAGAAATCATACGCGGCAGGACCAAGCAACATGG |
| | TCAGTTTTCTCTTGCAGTCGTCAGCCTGAACATAACATCCTTGG |
| | GATTACGCTCCCTCAAGGAGATAAGTGATGGAGATGTGATAA |
| | TTTCAGGAAACAAAAATTTGTGCTATGCAAATACAATAAACTG |
| | GAAAAAACTGTTTGGGACCTCCGGTCAGAAAACCAAAATTAT |
| | AAGCAACAGAGGTGAAAACAGCTGCAAGGCCACAGGCCAGGT |
| | CTGCCATGCCTTGTGCTCCCCCGAGGGCTGCTGGGGCCCGGAG |
| | CCCAGGGACTGCGTCTCTTGCCGGAATGTCAGCCGAGGCAGG |
| | GAATGCGTGGACAAGTGCAACCTTCTGGAGGGTGAGCCAAGG |
| | GAGTTTGTGGAGAACTCTGAGTGCATACAGTGCCACCCAGAGT |
| | GCCTGCCTCAGGCCATGAACATCACCTGCACAGGACGGGGAC |
| | CAGACAACTGTATCCAGTGTGCCCACTACATTGACGGCCCCCA |
| | CTGCGTCAAGACCTGCCCGGCAGGAGTCATGGGAGAAAACAA |
| | CACCCTGGTCTGGAAGTACGCAGACGCCGGCCATGTGTGCCAC |
| | CTGTGCCATCCAAACTGCACCTACGGATGCACTGGGCCAGGTC |
| | TTGAAGGCTGTCCAACGAATGGGCCTAAGATCCCGTCCATCGC |
| | CACTGGGATGGTGGGGGCCCTCCTCTTGCTGCTGGTGGTGGCC |
| | CTGGGGATCGGCCTCTTCATGTGA |
| pJ04043- | MLLLVTSLLLCELPHPAFLLIPQVQLVQSGAEVKKPGASVKVSCK |
| CD33scFv-VH- | ASGYTFTNYDINWVRQAPGQGLEWIGWIYPGDGSTKYNEKFKA |
| VL-CD8hinge- | KATLTADTSTSTAYMELRSLRSDDTAVYYCASGYEDAMDYWGQ |
| tm-41BB-zeta- | GTTVTVSSGGGGSGGGGSGGGGSDIQMTQSPSSLSASVGDRVTIN |
| T2A-EGFRt- | CKASQDINSYLSWFQQKPGKAPKTLIYRANRLVDGVPSRFSGSGS |
| epHIV72 | GQDYTLTISSLQPEDFATYYCLQYDEFPLTFGGGTKVEIKESKYGP |
| SEQ ID NO: 37 | AKPTTTPAPRPPTPAPTIASQPLSLRPEACRPAAGGAVHTRGLDFA |
| | CDIYIWAPLAGTCGVLLLSLVITKRGRKKLLYIFKQPFMRPVQTT |
| | QEEDGCSCRFPEEEEGGCELRVKFSRSADAPAYQQGQNQLYNEL |
| | NLGRREEYDVLDKRRGRDPEMGGKPRRKNPQEGLYNELQKDK |
| | MAEAYSEIGMKGERRRGKGHDGLYQGLSTATKDTYDALHMQA |
| | LPPRLEGGGEGRGSLLTCGDVEENPGPRMLLLVTSLLLCELPHPA |
| | FLLIPRKVCNGIGIGEFKDSLSINATNIKHFKNCTSISGDLHILPVAF |
| | RGDSFTHTPPLDPQELDILKTVKEITGFLLIQAWPENRTDLHAFEN |
| | LEIIRGRTKQHGQFSLAVVSLNITSLGLRSLKEISDGDVIISGNKNL |
| | CYANTINWKKLFGTSGQKTKIISNRGENSCKATGQVCHALCSPEG |
| | CWGPEPRDCVSCRNVSRGRECVDKCNLLEGEPREFVENSECIQC |
| | HPECLPQAMNITCTGRGPDNCIQCAHYIDGPHCVKTCPAGVMGE |
| | NNTLVWKYADAGHVCHLCHPNCTYGCTGPGLEGCPTNGPKIPSI |
| | ATGMVGALLLLLVVALGIGLFM |
| CD8a hinge and | GPAKPTTTPAPRPPTPAPTIASQPLSLRPEACRPAAGGAVHTRGLD |
| TM | FACDIYIWAPLAGTCGVLLLSLVIT |
| SEQ ID NO: 38 | |

### Certain CARs Specific for CD33M (CD33 Iso-L Protein or Long Isoform)

Some embodiments of the methods and compositions provided herein include a CAR specific for CD33M (CD33 Iso-L protein or long isoform). In some such embodiments, the CAR is encoded by a polynucleotide provided herein. The CAR can be encoded by the polynucleotide of any one of the alternatives herein or the vector of any one of the alternatives herein. The CAR can comprise an antibody or binding fragment thereof or a scFv, wherein the antibody or binding fragment thereof or scFv has a specific binding affinity for CD33M, an extracellular spacer, such as a de-immunized extracellular spacer, a transmembrane domain, a signaling domain, wherein the signaling domain comprises a 4-11B1 domain and/or CD3-zeta domain and a linker. In some embodiments, the antibody or binding fragment comprises a sequence set forth in SEQ ID NO:03 or SEQ ID NO:04. In some embodiments, the spacer comprises a IgG4 hinge or portion thereof (e.g., at least, equal to 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, or 55 amino acids or a length within a range defined by any two of the aforementioned lengths).

### Certain Cells for CAR T Cell Expression and Therapy

Some embodiments of the methods and compositions provided herein include a cell comprising a CAR as described in any one or more of the embodiments herein. In some embodiments, the cell is from a donor that is related or unrelated to the patient in need of CAR T cell therapy. In some embodiments, the cell is allogeneic. In some embodiments, the cell is from the patient in need of therapy. In some embodiments, the cell is a CD8+ T cytotoxic lymphocyte cell selected from the group consisting of naïve CD8+ T-cells, CD8+ memory T-cells, central memory CD8+ T-cells, regulatory CD8+ T-cells, IPS derived CD8+ T-cells, effector memory CD8+ T-cells and bulk CD8+ T-cells. In some embodiments, the cell is a CD4+ T helper lymphocyte cell that is selected from the group consisting of naïve CD4+ T-cells, CD4+ memory T-cells, central memory CD4+ T-cells, regulatory CD4+ T-cells, IPS derived CD4+ T-cells, effector memory CD4+ T-cells and bulk CD4+ T-cells. In some embodiments, the cell is a hematopoietic stem cell. In some embodiments, the cell further comprises a vector comprising a sequence encoding a soluble protein into said cell. In some embodiments, the soluble protein is a homeostatic cytokine. In some embodiments, the homeostatic cytokine is IL2, IL7, IL12 or IL15.

### Certain Methods of Making Donor Cells and Methods for Treatment

Some embodiments of the methods and compositions provided herein include methods of making a donor cell. In some embodiments, a method of making a donor cell comprising a CAR specific for CD33M (CD33 Iso-L protein or long isoform) of any one of the alternatives herein is provided. Some embodiments include identifying or selecting a subject homozygous for CD33m (CD33 Iso-S protein or short isoform) expression, for example by clinical or diagnostic evaluation, obtaining a cell from the subject, introducing the vector of any one of the alternatives described herein, into the cell, expanding the cell and isolating the cell. In some embodiments, the donor cell may be from a family member or non-family member. In some embodiments, the cell may be from the patient in need of therapy. In some embodiments, the cell is allogeneic.

Methods for determination of the phenotype of the patient (e.g., in the selecting or identifying step) in order to determine the CD33 isoform is appreciated by those of skill in the art and can be determined by known standard genetic testing.

In some embodiments, the cell is a CD8+ T cytotoxic lymphocyte cell selected from the group consisting of naïve CD8+ T-cells, CD8+ memory T-cells, central memory CD8+ T-cells, regulatory CD8+ T-cells, IPS derived CD8+ T-cells, effector memory CD8+ T-cells and bulk CD8+ T-cells. In some embodiments, the cell is a CD4+ T helper lymphocyte cell that is selected from the group consisting of naïve CD4+ T-cells, CD4+ memory T-cells, central memory CD4+ T-cells, regulatory CD4+ T-cells, IPS derived CD4+ T-cells, effector memory CD4+ T-cells and bulk CD4+ T-cells. In some embodiments, the cell is a hematopoietic stem cell.

In some embodiments, the method further comprises introducing a vector comprising a sequence encoding a soluble protein into said cell. In some embodiments, the soluble protein is a homeostatic cytokine. In some embodiments, the homeostatic cytokine is IL2, IL7, IL12 or IL15. In some embodiments, the method further comprises stimulating the cell. In some embodiments, stimulating comprises contacting the cell with anti-CD3 and/or anti-CD28 beads.

In some embodiments, a patient can be infused with donor cells that carry CD33m (CD33 Iso-S protein or short isoform) to prevent B cell aplasia. The donor cells that carry CD33m will not be recognized by the CD33M (CD33 Iso-L protein or long isoform) CAR which is specific for binding the IgV domain of CD33M.

Some embodiments of the methods and compositions provided herein include identifying a subject homozygous for CD33M (CD33 Iso-L protein or long isoform) expression or heterozygous for CD33M/CD33m expression, for example by clinical or diagnostic testing, obtaining a cell from the subject; gene-editing the cell to manufacture a homozygous CD33m (CD33 Iso-S protein or short isoform) genotype cell, introducing the vector of any one of the alternatives herein into the homozygous CD33m genotype cell, expanding the homozygous CD33m genotype cell and isolating the homozygous CD33m genotype cell. In some embodiments, the cell is a CD8+ T cytotoxic lymphocyte cell selected from the group consisting of naïve CD8+ T-cells, CD8+ memory T-cells, central memory CD8+ T-cells, regulatory CD8+ T-cells, IPS derived CD8+ T-cells, effector memory CD8+ T-cells and bulk CD8+ T-cells. In some embodiments, the cell is a CD4+ T helper lymphocyte cell that is selected from the group consisting of naïve CD4+ T-cells, CD4+ memory T-cells, central memory CD4+ T-cells, regulatory CD4+ T-cells, IPS derived CD4+ T-cells, effector memory CD4+ T-cells and bulk CD4+ T-cells. In some embodiments, the cell is a hematopoietic stem cell. In some embodiments, the cell is allogeneic. In some embodiments, the method further comprises introducing a vector comprising a sequence encoding a soluble protein into said cell. In some embodiments, the soluble protein is a homeostatic cytokine. In some embodiments, the homeostatic cytokine is IL2, IL7, IL12 or IL15. In some embodiments, the gene-editing is performed with a zinc finger nuclease, TALEN, CRISPR-CAS system or MegaTalen. In some embodiments, the method further comprising stimulating the cell. In some embodiments, stimulating comprises contacting the cells with anti-CD3 or anti-CD28 beads or both or a mixture of both.

### Certain Methods of Therapy

Some embodiments of the methods and compositions provided herein include methods of therapy, such as methods of treating, inhibiting or ameliorating a cancer in a subject. Some embodiments include identifying a subject having cancer, wherein the subject is homozygous for CD33M (CD33 Iso-L protein or long isoform) expression or heterozygous for CD33M/CD33m expression, ablating cancer cells or bone marrow from the subject, providing a donor cell that is homozygous for CD33m (CD33 Iso-S protein or short isoform) expression for hematopoietic stem cell transplantation by allogeneic transplant, monitoring the subject for engraftment; and providing the CD33m genotype cell comprising a chimeric antigen receptor specific for CD33M of any one of the alternatives herein or a cell manufactured by any one of the alternatives herein.

Monitoring of a patient for engraftment is known to those of skill in the art. Engraftment involves the process in which the transplanted stem cells find their way to the bone marrow spaces of the large bones of the body. Blood tests are performed to test the white blood cell count which should rise as a first indicator of engraftment.

In some embodiments, the donor cell is a hematopoietic stem cell homozygous for CD33m (CD33 Iso-S protein or short isoform). In some embodiments, the subject has acute myeloid leukemia.

In some embodiments, the method further comprises providing a drug to induce expression of a chimeric antigen receptor specific for CD33M (CD33 Iso-L protein or long isoform). In some embodiments, the drug is tamoxifen.

In some embodiments, the ablation is performed by chemotherapy and/or radiation therapy.

In some embodiments, the subject is monitored for side effects that are indicative of cytokine storm or cerebral edema. In some embodiments, wherein the subject is experiencing side effects, drugs that turn on suicide genes in the CAR T cells are administered.

In another alternative in the method of treating, inhibiting or ameliorating a cancer in a subject, the method comprises identifying a subject having cancer, e.g., by clinical or diagnostic evaluation, wherein the subject is homozygous for CD33M (CD33 Iso-L protein or long isoform) expression or heterozygous for CD33M/CD33m expression; providing a donor cell, wherein the donor cell is homozygous for CD33M expression or heterozygous CD33M/CD33m expression; gene-editing the donor cell to convert the donor cell to a homozygous CD33m (CD33 Iso-S protein or short isoform) genotype, thereby making a CD33m genotype donor cell; ablating cancer cells or bone marrow from the subject; providing the CD33m genotype donor cell to the subject in need by allogeneic transplant; monitoring the subject for engraftment; and providing the cell comprising the chimeric antigen receptor specific for CD33M of any one of the alternatives herein or a cell manufactured by any one of the alternatives herein. In some embodiments, the donor cell is a hematopoietic stem cell. In some embodiments, the subject has acute myeloid leukemia, chronic myelogenous leukemia or chronic granulocytic leukemia. In some embodiments, the gene-editing is performed with a zinc finger nuclease, TALEN, CRISPR-CAS system or MegaTalen. In some embodiments, the method further comprises providing a drug to induce expression of a chimeric antigen receptor specific for CD33M. In some embodiments, the drug is tamoxifen. In some embodiments, the ablation is performed by chemotherapy and/or radiation therapy. In some embodiments, the subject is monitored for side effects that are indicative of cytokine storm or cerebral edema. In some embodiments, wherein the subject is experiencing side effects, drugs that turn on suicide genes in the CAR T cells are administered.

In another alternative method of treating, inhibiting or ameliorating a leukemia in a subject, the method comprises a) identifying a subject having leukemia, e.g., by clinical or diagnostic evaluation, wherein the subject is homozygous for CD33M (CD33 Iso-L protein or long isoform) expression or heterozygous for CD33M/CD33m expression; b) extracting cells from the subject, thereby providing extracted cells; c) gene-editing the extracted cells to convert the extracted cells to a homozygous CD33m (CD33 Iso-S protein or short isoform) genotype, thereby making CD33m genotype cells; d) splitting the CD33m genotype cells, wherein the CD33m genotype cells are split into a first fraction and a second fraction, wherein the first fraction comprises cells selected for autologous transplant and the second fraction comprises cells selected for generating chimeric antigen receptor bearing cells; e) introducing the vector of any one of the alternatives herein into the second fraction of cells, thereby generating chimeric antigen receptor bearing cells specific for CD33M; f) ablating leukemic cells or bone marrow from the subject; g) administering the CD33m genotype cells of step c) to the subject by an autologous transfer technique; h) monitoring the subject for engraftment; and i) administering the chimeric antigen receptor bearing cells of step e) to the subject by autologous transfer for removal of residual CD33M bearing cells. In some embodiments, the subject has acute myeloid leukemia. In some embodiments, the gene-editing is performed with a zinc finger endonuclease, TALEN, CRISPR-CAS system or MegaTalen. In some embodiments, the cell is a hematopoietic stem cell. In some embodiments, the method further comprises providing a drug to induce expression of a chimeric antigen receptor specific for CD33M. In some embodiments, the drug is tamoxifen. In some embodiments, the ablation is performed by chemotherapy and/or radiation therapy. In some embodiments, the subject is monitored for side effects that are indicative of cytokine storm or cerebral edema. In some embodiments, wherein the subject is experiencing side effects, drugs that turn on suicide genes in the CAR T cells are administered.

In another alternative for treating, inhibiting or ameliorating a leukemia in a subject, the method comprises a) identifying a subject having leukemia, wherein the subject is homozygous for CD33M (CD33 Iso-L protein or long isoform) expression or heterozygous for CD33M/CD33m expression, e.g., by clinical or diagnostic evaluation; b) extracting cells from the subject, thereby providing extracted cells; c) gene-editing the extracted cells to convert the extracted cells to a homozygous CD33m genotype, thereby generating CD33m (CD33 Iso-S protein or short isoform) genotype cells; d) ablating leukemic cells or bone marrow from the subject; e) administering the CD33m genotype cells of step c) to the subject by an autologous transfer technique; f) monitoring the subject for engraftment; and g) administering the chimeric antigen receptor bearing cells of any one of the alternatives herein to the subject by an allogeneic transfer technique. In some embodiments, the subject has acute myeloid leukemia. In some embodiments, the gene-editing is performed with a zinc finger endonuclease, TALEN, CRISPR-CAS system or MegaTalen. In some embodiments, the cell is a hematopoietic stem cell. In some embodiments, the method further comprises providing a drug to induce expression of a chimeric antigen receptor specific for CD33M. In some embodiments, the drug is tamoxifen. In some embodiments, the ablation is performed by chemotherapy and/or radiation therapy.

### EXAMPLES

### Example 1—Preparation of CAR T-Cells

Various polynucleotides encoding CD33 targeting CARs were constructed. As shown in FIG. 5, polynucleotides encoding CD33 specific CARs included: an EF1p promoter, a leader sequence, one of a number of single-chain variable fragment (scFv) that specifically recognized an epitope of CD33; one of various spacer domains (marked “L” long, “M” medium, “CD8a”, and “S” short); a transmembrane domain derived from a human CD28 (CD28tm); a costimulatory domain derived from human 4-1BB; a CD3ζ-derived signaling domain; a T2A ribosomal skip sequence; and a truncated EGFR (EGFRt) transduction marker; and optionally a further T2A ribosomal skip sequence and a dihydrofolate reductase double mutant (DHFRdm) transgene configured for methotrexate selection. The various scFvs included a VH-linker-VL configuration, and a VL-linker-VH configuration. The various spacer domains included a IgG4-CH2-CH3 “L”; a IgG4-CH3 “M”; a CD8 alpha “CD8a”; and a hinge “S”. The polynucleotides were constructed in lentiviral vectors.

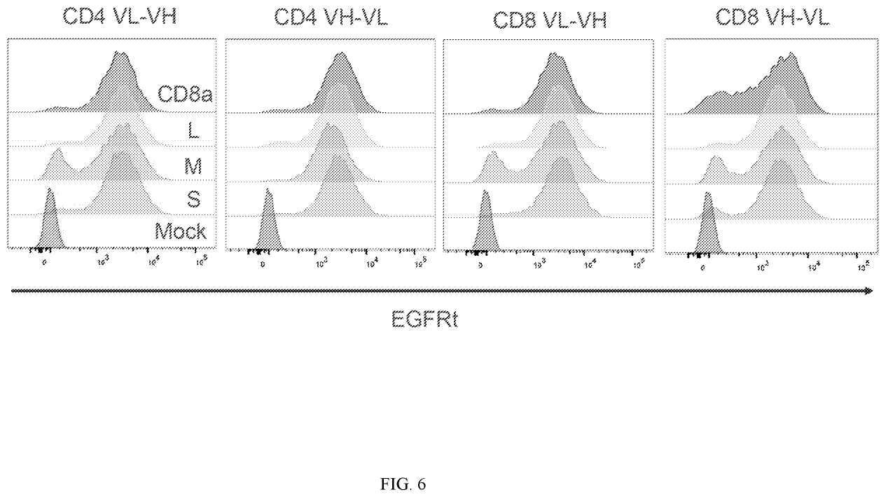

CD4+ and CD8+ T cells were purified from PBMC using CD4 or CD8 microbeads. Cells were transduced with the lentiviral vectors containing the various polynucleotides encoding CD33 specific CARs. Cells expressing the EGFRt marker were immunomagnetically purified using biotinylated Erbitux and anti-biotin microbeads. FIG. 6 is a flow cytometry analysis of either CD4+ T cells or CD8+ T cells transduced with various polynucleotides encoding CD33 targeting CARs and shows that CAR-expressing T cells were efficiently purified from the various transduced populations.

### Example 2—In Vitro Cytolytic Activity of CAR T Cells

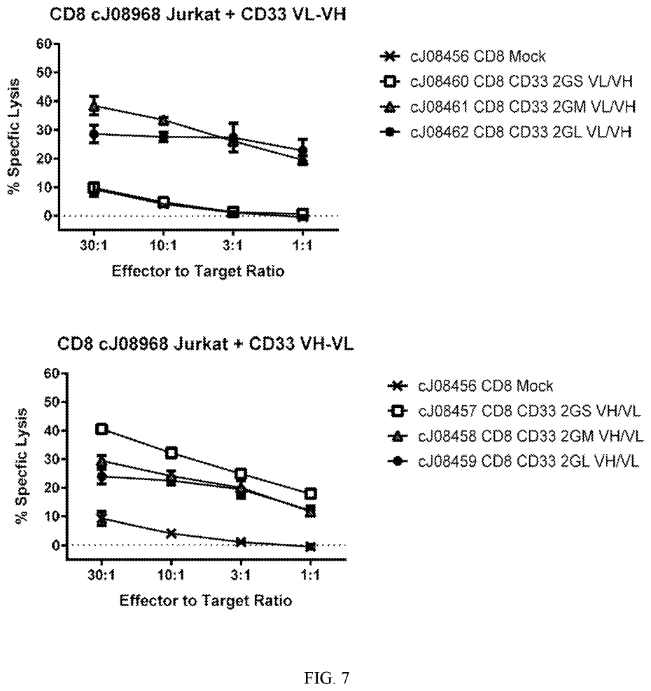

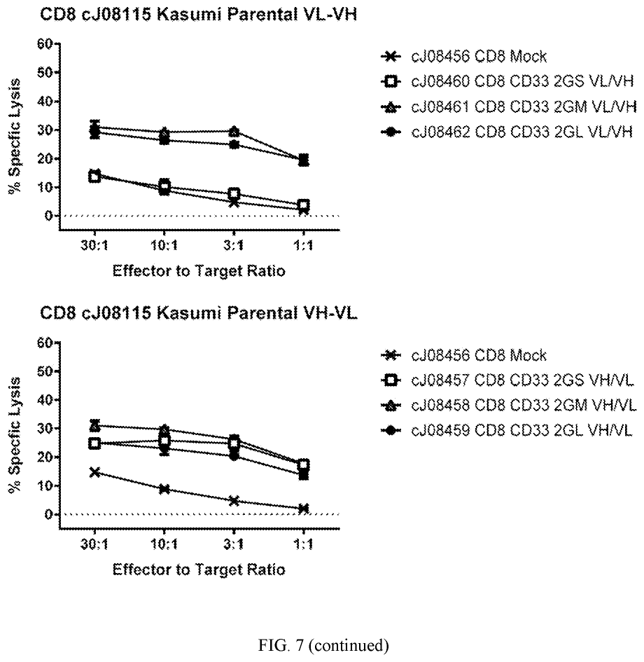

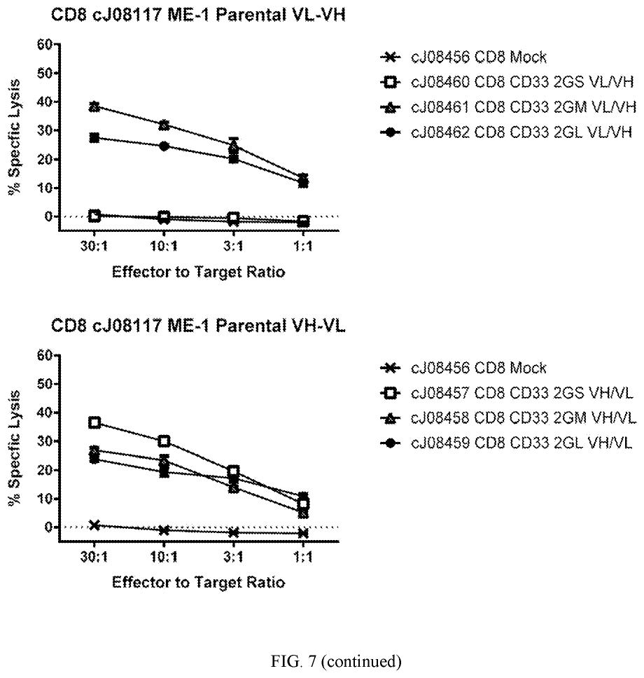

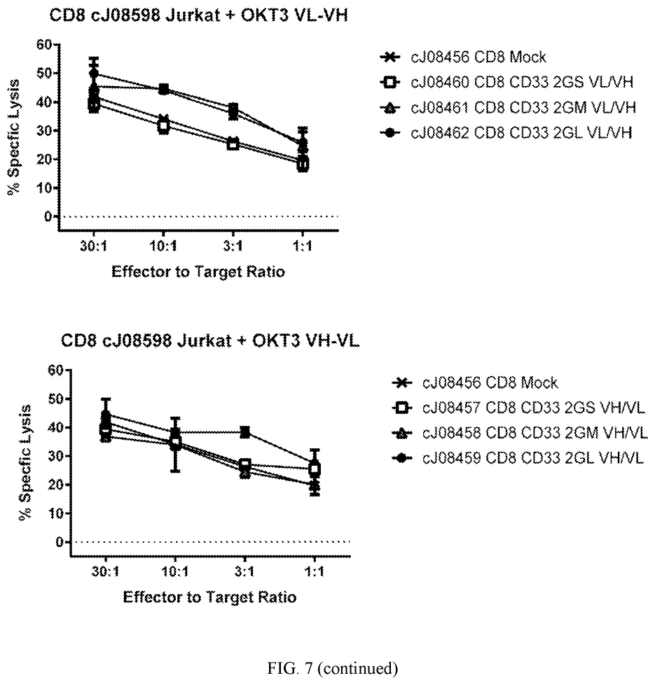

Cytolytic activity of CD8+ T cells containing various CD33 targeting CARs was examined. Effector CAR T cells were incubated in a 4-hour chromium release assay at various ratios with target CD33+ cells. Effector:target ratios 30:1, 10:1, 3:1 or 1:1. Target CD33+ cells included Jurkat cells, Kasumi cells, ME-1 cells (FI, and Jurkat OKT3 cells as a positive control for T cell activation (FIG. 7).

CD8+ T cells containing a CD33 targeting CAR having a VL/VH scFv and a short spacer was unable to elicit cytolytic activity, while CD8+ T cells containing a CD33 targeting CAR having a VL/VH scFv and either a medium or long spacer elicited similar cytolytic activities (FIG. 7). CD8+ T cells containing a CD33 targeting CAR having a VH/VL scFv and a short spacer elicited the most specific lysis compared to CD8+ T cells containing a CD33 targeting CAR having a VH/VL scFv and either a medium or long spacer (FIG. 7).

### Example 3—In Vitro Induction of Cytokines by CAR T Cells

A cytokine release assay was performed with CD4+ T cells containing various CD33 targeting CARs. CAR T cells were incubated for 24 hours with CD33+ cells. Cytokines released into the supernatants were measured. CD33+ cells included Jurkat parental cells, Jurkat OKT3 cells, Jurkat CD33 cells, Kasumi parental cells, and ME-1 cells.

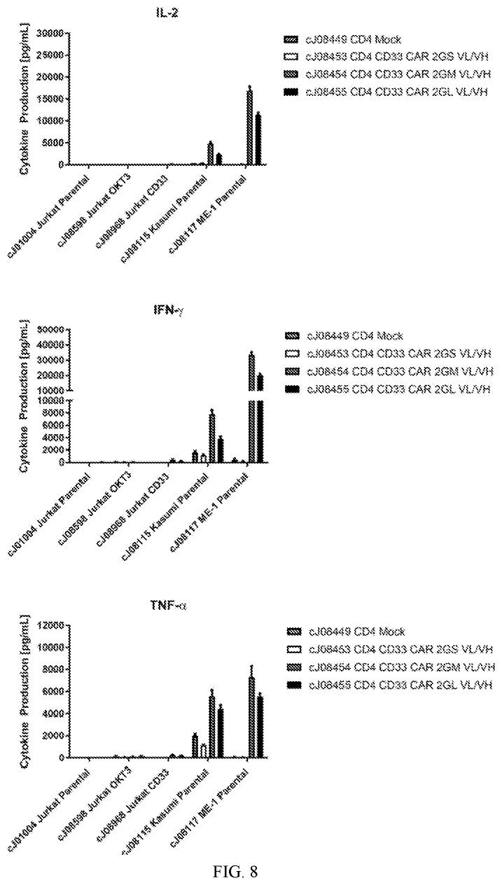

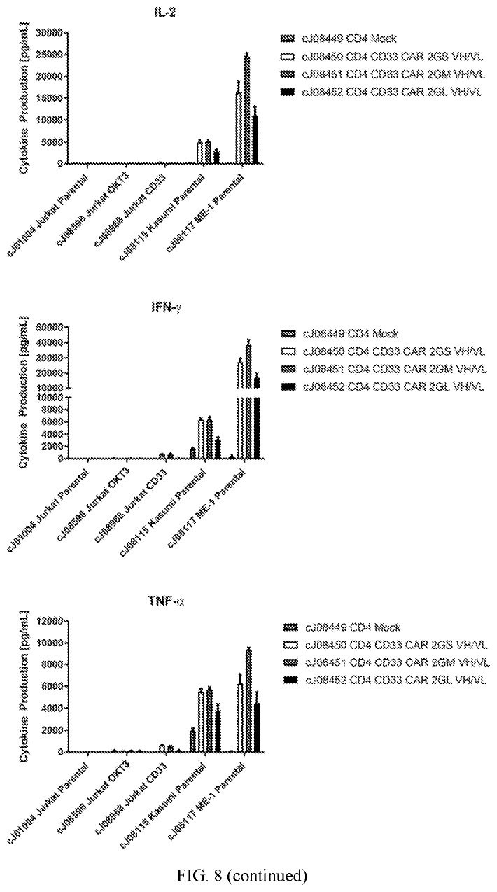

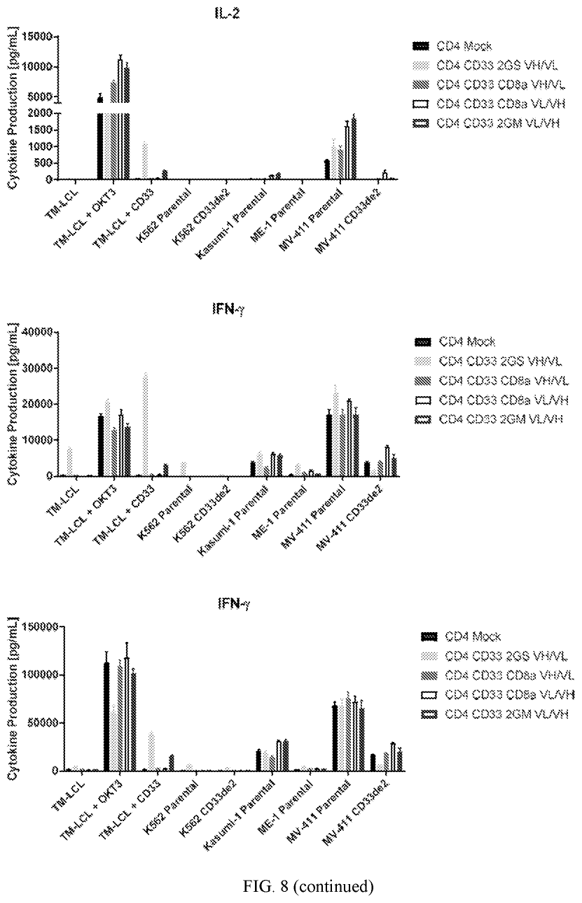

CD4+ T cells containing a CD33 targeting CAR having a VL/VH scFv and a short spacer was unable to elicit a significant cytokine response against CD33+ tumor targets. CD4+ T cells containing a CD33 targeting CAR having a VL/VH scFv and either a medium or long spacer induced cytokines to be released, and cells containing a CAR with the medium spacer consistently outperformed cells containing a CAR with the long spacer for IL2, IFN-γ, and TNF-α production (FIG. 8). CD4+ T cells containing a CD33 targeting CAR having a VH/VL scFv and either the short spacer or medium spacer induced similar levels of cytokine production upon co-culture with CD33+ tumor targets (FIG. 8). CD4+ T cells containing a CD33 targeting CAR having a VH/VL scFv and a long spacer induced lower levels of cytokines than cells containing a CAR with either the medium spacer or short spacer.

CD4+ T cells containing a CD33 targeting CAR having either a VH/VL or VL/VH scFv and a CD8a spacer, elicited similar levels of cytokines to CD33 targeting CAR having either the short spacer or medium spacer (FIG. 8).

### Example 4—In Vivo Activity of CAR T Cells

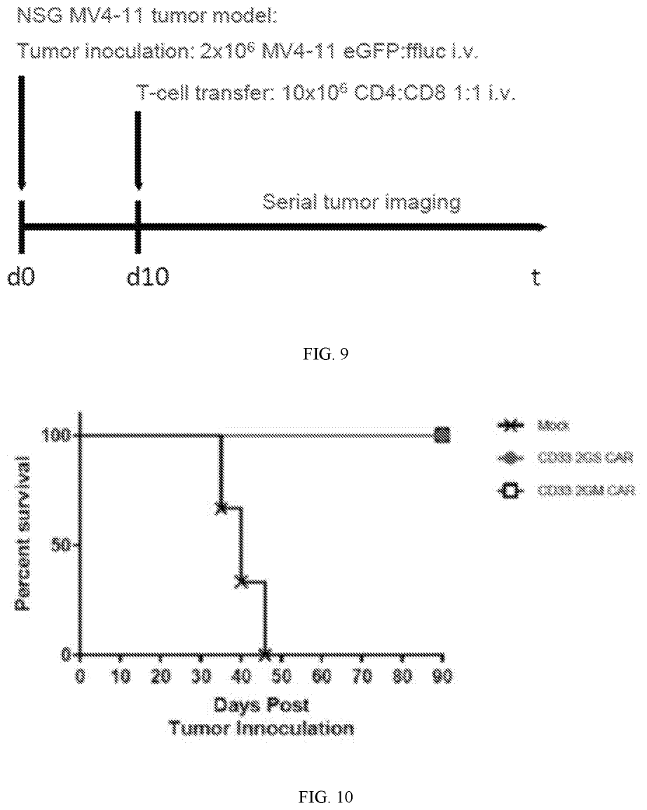

A xenograft model of AML was used to test in vivo activity of CAR T cells containing either a CD33 targeting CAR having a VH/VL scFv and a short spacer, or a CD33 targeting CAR having a VL/VH scFv and a medium spacer. Briefly, cohorts of mice were inoculated with 2×10⁶ eGFP:ffluc expressing MV4-11 intravenously (i.v.) at Day 0 and 10×10⁶ CD33-CAR expressing CD4:CD8 at a 1:1 ratio i.v. at Day 10 (FIG. 9). Serial bioluminescence tumor imaging was performed weekly up to 90 days.

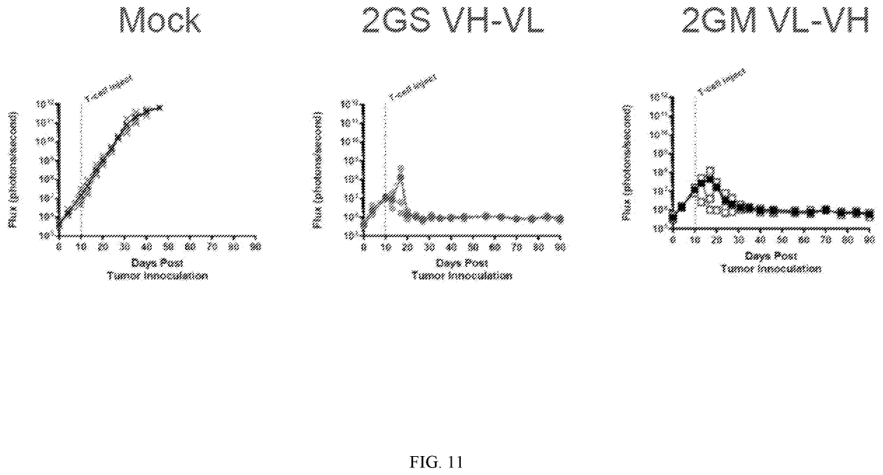

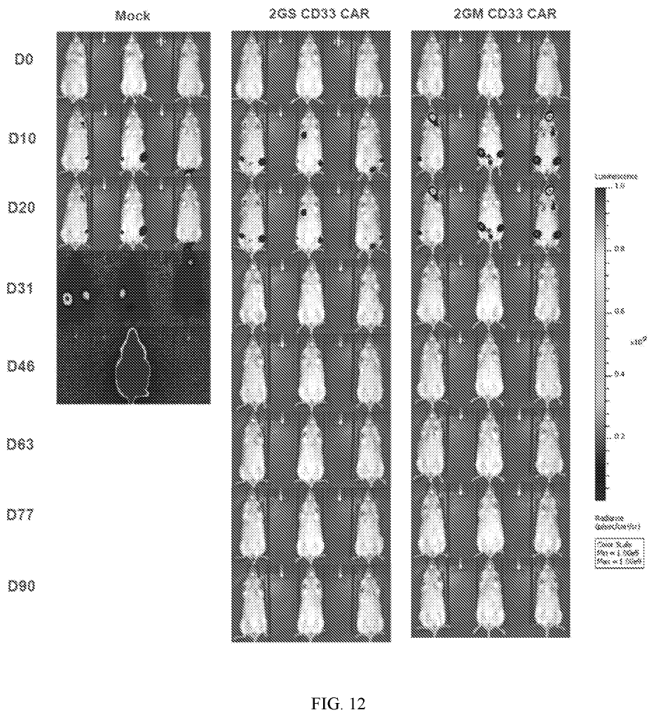

Kaplan-Meier analysis of survival for individual treatment and control groups (FIG. 10). Total flux of individual mice per treatment group was measured up to 90 days post tumor inoculation (FIG. 11). Representative bioluminescent images of mice prior to and following CD33CAR treatment until end of study (FIG. 12). Treatment with either CAR T cells containing a CD33 targeting CAR having a VH/VL scFv and a short spacer, or CAR T cells containing a CD33 targeting CAR having a VL/VH scFv and a medium spacer increased survival in mice over control, and significantly reduced tumor volume over control.

The term “comprising” as used herein is synonymous with “including,” “containing,” or “characterized by,” and is inclusive or open-ended and does not exclude additional, unrecited elements or method steps.

The above description discloses several methods and materials of the present invention. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

1. A nucleic acid encoding a chimeric antigen receptor (CAR), wherein the CAR comprises:
a ligand binding domain capable of or configured to specifically bind to a long isoform of a CD33 protein (CD33M);
a spacer;
a transmembrane domain; and
an intracellular signaling domain.

2-79. (canceled)
